(19) United States
(12) Patent Application Publication
(10) Pub. No.: US 2015/0361063 A1
(43) Pub. Date: Dec. 17, 2015

(54) TREATMENT OF AMYLOIDOSIS BY COMPOUNDS THAT REGULATE RETROMER STABILIZATION

(71) Applicants: Brandeis University, Waltham, MA (US); The Trustees of Columbia University in the City of New York, New York, NY (US)

(72) Inventors: Dagmar Ringe, Cambridge, MA (US); Gregory A. Petsko, New York, NY (US); Scott A. Small, Millerton, NY (US); Vincent Mecozzi, Boston, MA (US)

(21) Appl. No.: 14/832,941

(22) Filed: Aug. 21, 2015

Related U.S. Application Data
(63) Continuation of application No. 14/362,095, filed on May 30, 2014.
(60) Provisional application No. 61/567,023, filed on Dec. 5, 2011.

(51) Int. Cl. C07D 333/18; A61K 31/381

(52) U.S. Cl. CPC C07D 333/18; A61K 31/381

(57) ABSTRACT

The present invention provides pharmaceutical compositions and related methods for stabilizing retromer in cells.

### CROSS-REFERENCE TO RELATED APPLICATIONS

This application claims the benefit of and priority to U.S. Provisional Application Ser. No. 61/567,023, filed Dec. 5, 2011, the entire contents of which are hereby incorporated by reference.

### BACKGROUND

Retromer is a protein complex peripherally associated with endosomal organelles and controls trafficking of a number of critical cellular cargo molecules within tubulovesicular carriers to the trans Golgi network (TGN). Defects in the function of retromer-mediated intracellular trafficking have been linked to the pathogenesis of certain diseases and disorders.

### SUMMARY

Among other things, the present invention encompasses the recognition that individuals affected by a condition associated with impaired trafficking and/or processing of certain cellular peptides exhibit reduced levels of the retromer protein complex in vivo due at least in part to reduced stability of the functional complex. The invention therefore provides pharmaceutical compositions that comprise one or more agents that stabilize the retromer complex. The invention also encompasses the finding that retromer complex contains several drug target sites which are formed at or near the interface of at least two components of the complex. In some embodiments, drug target sites on retromer are localized at the interface between VPS35 and VPS29. In some embodiments, retromer-stabilizing compositions of the invention exert their stabilizing effect on fully assembled retromer complex, but not on individual protein components.

### BRIEF DESCRIPTION OF THE DRAWING

FIG. 1. Graph depicting the effect of compound 55712, [5-(carbamimidoylsulfanylmethyl)thiophen-2-yl]methyl carbamimidothioate on stabilization of the retromer complex.

FIG. 2. Depiction of binding interaction of retromer with thiophene-2,5-diylbis(methylene)dicarbamimidothioate dihydrochloride.

### DEFINITIONS

Amyloid: Amyloids are insoluble fibrous protein aggregates sharing specific structural traits. Abnormal accumulation of amyloid in organs may lead to amyloidosis, and may play a role in various pathological conditions, including neurodegenerative diseases. In the context of the present invention, relevant amyloids are those requiring retromer-mediated protein sorting/trafficking for proper cellular processing and/or regulation.”

Amyloidosis: The term “amyloidosis,” as used herein, refers to a condition characterized by abnormal trafficking and/or processing that results in accumulation of one or more amyloidogenic peptides which can cause a disease or disorder associated with the amyloid (e.g., “amyloidopathy”). Examples of amyloidosis may include, without limitation: Alzheimer's disease (AD); Diabetes mellitus type 2; Parkinson's disease (PD); Transmissible spongiform encephalopathy (e.g., Bovine spongiform encephalopathy); Huntington's Disease (HD); Medullary carcinoma of the thyroid; Cardiac arrhythmias, Isolated atrial amyloidosis; Atherosclerosis; Rheumatoid arthritis; Aortic medial amyloid; Prolactinomas; Familial amyloid polyneuropathy; Hereditary non-neuropathic systemic amyloidosis; Dialysis related amyloidosis; Finnish amyloidosis; Lattice corneal dystrophy; Cerebral amyloid angiopathy; Cerebral amyloid angiopathy (Icelandic type); systemic AL amyloidosis and Sporadic Inclusion Body Myositis.

Amyloidogenic protein: As used herein, the term “amyloidogenic proteins” refers to ertain cellular proteins that confer propensity to form toxic amyloids under certain conditions. Examples of amyloidogenic proteins include, without limitation: Beta amyloid; IAPP (Amylin); Alpha-synuclein; PrPSc; Huntingtin; Calcitonin; Atrial natriuretic factor; Apolipoprotein AI; Serum amyloid A; Medin; Prolactin; Transthyretin; Lysozyme; Beta 2 microglobulin; Gelsolin; Keratoepithelin; Beta amyloid; Cystatin; Immunoglobulin light chain AL and S-IBM.

Aryl: The term “aryl” used alone or as part of a larger moiety as in “aralkyl,” “aralkoxy,” or “aryloxyalkyl,” refers to monocyclic, bicyclic, and tricyclic ring systems having a total of five to fourteen ring members, wherein one or more ring in the system is aromatic and wherein each ring in the system contains 3 to 7 ring members. The term “aryl” may be used interchangeably with the term “aryl ring”. The term “aryl” also refers to heteroaryl ring systems as defined hereinbelow. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Aliphatic: The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-20 aliphatic carbon atoms. In some embodiments, aliphatic groups contain 1-6 aliphatic carbon atoms. In yet other embodiments aliphatic groups contain 1-4 aliphatic carbon atoms. In some embodiments, “cycloaliphatic” (or “carbocycle” or “cycloalkyl”) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl. In other embodiments, an aliphatic group may have two geminal hydrogen atoms replaced with oxo (a bivalent carbonyl oxygen atom ═O), or a ring-forming substituent, such as —O-(straight or branched alkylene or alkylene)-O— to form an acetal or ketal.

In certain embodiments, exemplary aliphatic groups include, but are not limited to, ethynyl, 2-propynyl, 1-propenyl, 2-butenyl, 1,3-butadienyl, 2-pentenyl, vinyl (ethenyl), allyl, isopropenyl, methyl, ethyl, propyl, isopropyl, butyl, isobutyl, sec-butyl, tert-butyl, pentyl, isopentyl, sec-pentyl, neo-pentyl, tert-pentyl, cyclopentyl, hexyl, isohexyl, sec-hexyl, cyclohexyl, 2-methylpentyl, tert-hexyl, 2,3-dimethylbutyl, 3,3-dimethylbutyl, 1,3-dimethylbutyl, and 2,3-dimethyl but-2-yl.

Alkylidene: The term “alkylidene,” as used herein, refers to a divalent group formed from an alkane by removal of two hydrogen atoms from the same carbon atom, the free valencies of which are part of a double bond. By way of nonlimiting example, an alkylidene may be of the formula ═C(R^{q})₂, ═CHR^{q}, or ═CH₂, wherein R^{q} represents any suitable substituent other than hydrogen.

RACE: The abbreviation “BACE,” as used herein, refers to Beta-secretase 1 (BACE1), which is also known as beta-site APP cleaving enzyme 1 (beta-site amyloid precursor protein cleaving enzyme 1), memapsin-2 (membrane-associated aspartic protease 2), and aspartyl protease 2 (ASP2). BASE is a protease that is known to cleave APP.

Combination therapy: The term “combination therapy,” as used herein, refers to those situations in which two or more different pharmaceutical agents are administered in overlapping regimens so that the subject is simultaneously exposed to both agents.

Determine: Many methodologies described herein include a step of “determining.” Those of ordinary skill in the art, reading the present specification, will appreciate that such “determining” can utilize any of a variety of techniques available to those skilled in the art, including, for example, specific techniques explicitly referred to herein. In some embodiments, a determination involves manipulation of a physical sample. In some embodiments, a determination involves consideration and/or manipulation of data or information, for example utilizing a computer or other processing unit adapted to perform a relevant analysis. In some embodiments, a determination involves receiving relevant information and/or materials from a source.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regiment, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses.

Haloalkyl: The terms “haloalkyl,” “haloalkenyl” and “haloalkoxy” means alkyl, alkenyl or alkoxy, as the case may be, substituted with one or more halogen atoms. The term “halogen” means F, Cl, Br, or I. Such “haloalkyl,” “haloalkenyl” and “haloalkoxy” groups may have two or more halo substituents which may or may not be the same halogen and may or may not be on the same carbon atom. Examples include chloromethyl, periodomethyl, 3,3-dichloropropyl, 1,3-difluorobutyl, trifluoromethyl, and 1-bromo-2-chloropropyl.

Heteroaryl: The term “heteroaryl,” used alone or as part of a larger moiety as in “heteroaralkyl” or “heteroarylalkoxy,” refers to monocyclic, bicyclic, and tricyclic ring systems having a total of five to fourteen ring members, wherein one or more ring in the system is aromatic, one or more ring in the system contains one or more heteroatoms, and wherein each ring in the system contains 3 to 7 ring members. The term “heteroaryl” may be used interchangeably with the term “heteroaryl ring” or the term “heteroaromatic”. Heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl.

The terms “heteroaryl” and “heteroar-,” as used herein, also include groups in which a heteroaromatic ring is fused to one or more aryl, cycloaliphatic, or heterocyclyl rings. Exemplary heteroaryl rings include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one.

Heteroatom: The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl).

Heterocycle: The term “heterocycle,” “heterocyclyl,” “heterocycloaliphatic,” or “heterocyclic” as used herein means non-aromatic, monocyclic, bicyclic, or tricyclic ring systems in which one or more ring members is an independently selected heteroatom. In some embodiments, the “heterocycle,” “heterocyclyl,” “heterocycloaliphatic,” or “heterocyclic” group has three to fourteen ring members in which one or more ring members is a heteroatom independently selected from oxygen, sulfur, nitrogen, or phosphorus, and each ring in the system contains 3 to 7 ring members.

A heterocyclic ring can be attached to its pendant group at any heteroatom or carbon atom that results in a stable structure and, when specified, any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl.

Optionally substituted: As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘}C(O)R^{∘}; —N(R^{∘})C(S)R^{∘}; —(CH₂)₀₋₄N(R^{∘}C(O)NR^{∘})₂; —N(R^{∘}C(S)NR^{∘})₂; —(CH₂)₀₋₄N(R^{∘}C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘}R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched)alkylene)O—N(R^{∘})₂; or —(C₁₋₄ straight or branched)alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R^{*}₂))₂₋₃O—, or —S(C(R*₂))₂₋₃S—, and ═C(R*)₂, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Patient: As used herein, the term “patient”, “subject”, or “test subject” refers to any organism to which provided PYT compound is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.). In some embodiments, a subject may be suffering from, and/or susceptible to a disease, disorder, and/or condition (e.g., a neurodegenerative disease, a disease, disorder or condition associated with protein aggregation, ALS, etc.).

Pharmaceutically acceptable salt: As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or a pharmaceutically active metabolite or residue thereof. As used herein, the term “pharmaceutically active metabolite or residue thereof” means that a metabolite or residue thereof is also a pharmaceutically active compound in accordance with the present invention.

Prevention: The term “prevention,” as used herein, refers to a delay of onset, and/or reduction in frequency and/or severity of one or more symptoms of a particular disease, disorder or condition (e.g., infection for example with influenza virus). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

Prodrug: A general, a “prodrug”, as that term is used herein and as is understood in the art, is an entity that, when administered to an organism, is metabolized in the body to deliver a therapeutic agent of interest. Various forms of “prodrugs” are known in the art. For examples of such prodrug derivatives, see:
a) Design of Prodrugs, edited by H. Bundgaard, (Elsevier, 1985) and Methods in Enzymology, 42:309-396, edited by K. Widder, et al. (Academic Press, 1985);
b) A Textbook of Drug Design and Development, edited by Krogsgaard-Larsen;
c) Bundgaard, Chapter 5 “Design and Application of Prodrugs”, by H. Bundgaard, p. 113-191 (1991);
d) Bundgaard, Advanced Drug Delivery Reviews, 8:1-38 (1992);
e) Bundgaard, et al., Journal of Pharmaceutical Sciences, 77:285 (1988);
and
f) Kakeya, et al., Chem. Pharm. Bull., 32:692 (1984).

Protein: As used herein, the term “protein” refers to a polypeptide (i.e., a string of at least two amino acids linked to one another by peptide bonds). In some embodiments, proteins include only naturally-occurring amino acids. In some embodiments, proteins include one or more non-naturally-occurring amino acids (e.g., moieties that form one or more peptide bonds with adjacent amino acids). In some embodiments, one or more residues in a protein chain contains a non-amino-acid moiety (e.g., a glycan, etc). In some embodiments, a protein includes more than one polypeptide chain, for example linked by one or more disulfide bonds or associated by other means. In some embodiments, proteins contain L-amino acids, D-amino acids, or both; in some embodiments, proteins contain one or more amino acid modifications or analogs known in the art. Useful modifications include, e.g., terminal acetylation, amidation, methylation, etc. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Retromer: Unless expressly stated, the term “retromer” as used herein refers to a multimeric complex (e.g., retromer complex) typically composed of two distinct sub-complexes, namely, in mammals, a core trimer composed of VPS35-VPS29-VPS26 (VPS: “vacuolar protein sorting”) and an associated homo or heterodimer of sorting nexin (SNX) proteins, containing combinations of SNX1, SNX2, SNX5 and SNX6. The core trimer composed of the VPS components is typically referred to as the “cargo recognition complex” or “cargo-binding complex.”

Retromer component: The phrase “retromer component,” as used herein, refers to an individual component (e.g., protein or subunit) that makes up the retromer complex. For example, VPS35, VPS29, VPS26, SNX1, SNX2, SNX5 and SNX6 are retromer components.

Retromer stabilizing agent: The phrase “retromer stabilizing agent,” as used herein, refers to a chemical entity that has an effect of stabilizing retromer complex. In some embodiments, a retromer stabilizing agent may function as a molecular chaperon. In some embodiments, a retromer stabilizing agent may “correct” a misfolded protein (e.g., retromer component) or protein complex (e.g., retromer) to facilitate or restore its intended function. Thus, in some embodiments, a retromer stabilizing agent may be conformational-specific. In some embodiments, a retromer stabilizing agent may have higher affinity or specificity for a misfolded target, as compared to its wild type counterpart, which is correctly folded.

Stable: The term “stable,” as used herein in reference to a compound, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

Secondary label: The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

Tautomeric forms: The phrase “tautomeric forms,” as used herein, is used to describe different isomeric forms of organic compounds that are capable of facile interconversion. Unless otherwise stated, all tautomeric forms of the compounds of the invention are within the scope of the invention. Tautomers may be characterized by the formal migration of a hydrogen atom or proton, accompanied by a switch of a single bond and adjacent double bond. In some embodiments, tautomers may result from prototropic tautomerism (i.e., the relocation of a proton). In some embodiments, tautomers may result from valence tautomerism (i.e., the rapid reorganization of bonding electrons). All such tautomeric forms are intended to be included within the scope of the present invention. In some embodiments, tautomeric forms of a compound exist in mobile equilibrium with each other, so that attempts to prepare the separate substances results in the formation of a mixture. In some embodiments, tautomeric forms of a compound are separable and isolatable compounds. In some embodiments of the invention, chemical compositions may be provided that are or include pure preparations of a single tautomeric form of a compound. In some embodiments of the invention, chemical compositions may be provided as mixtures of two or more tautomeric forms of a compound. In certain embodiments, such mixtures contain equal amounts of different tautomeric forms; in certain embodiments, such mixtures contain different amounts of at least two different tautomeric forms of a compound. In some embodiments of the invention, chemical compositions may contain all tautomeric forms of a compound. In some embodiments of the invention, chemical compositions may contain less than all tautomeric forms of a compound. In some embodiments of the invention, chemical compositions may contain one or more tautomeric forms of a compound in amounts that vary over time as a result of interconversion. In some embodiments of the invention, the tautomerism is keto-enol tautomerism. One of skill in the chemical arts would recognize that a keto-enol tautomer can be “trapped” (i.e., chemically modified such that it remains in the “enol” form) using any suitable reagent known in the chemical arts in to provide an enol derivative that may subsequently be isolated using one or more suitable techniques known in the art. Unless otherwise indicated, the present invention encompasses all tautomeric forms of relevant compounds, whether in pure form or in admixture with one another.

Therapeutic agent: As used herein, the phrase “therapeutic agent” refers to any agent that elicits a desired biological or pharmacological effect.

Thermal stability: The phrase “thermal stability,” as used herein, refers to a measure of stability of a molecule (e.g., a complex) in correlation with temperature.

Treatment: As used herein, the term “treatment” refers to any method used to alleviate, delay onset, reduce severity or incidence, or yield prophylaxis of one or more symptoms or aspects of a disease, disorder, or condition. For the purposes of the present invention, treatment can be administered before, during, and/or after the onset of symptoms.

Type 1 transmembrane protein: The phrase “type 1 transmembrane proteins,” as used herein, refers to single-pass transmembrane proteins which have their N-terminus exposed to the extracellular or luminal space.

Unit dose: The expression “unit dose” as used herein refers to a physically discrete unit of a pharmaceutical composition, formulated for administration to a subject. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple doses is required, or expected to be required, in order to achieve an intended effect. The unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may contain a variety of components in addition to the therapeutic agent(s). For example, acceptable carriers (e.g., pharmaceutically acceptable carriers), diluents, stabilizers, buffers, preservatives, etc., may be included as described infra. It will be understood, however, that the total daily usage of a formulation of the present disclosure will often be decided by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated,” as used herein, means that a moiety has one or more units of unsaturation. As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

VPS35: Vacuolar protein sorting-associated protein 35 (i.e., VPS35), is a protein that in humans is encoded by the VPS35 gene, which belongs to a group of vacuolar protein sorting (VPS) genes. The wild type VPS35 protein is a 796 amino acid polypeptide (SEQ ID NO: 1) with an approximate molecular weight of ˜92 kDa.

VPS10 domain: The phrase “VPS10 domain,” as used herein, refers to a peptide sequence or conformational specific module of a protein similar to that found on the yeast VPS10 protein. A VPS10 domain is recognized as a cargo for retromer-dependent protein transport. Thus, cellular trafficking of proteins containing a VPS10 domain may be mediated by retromer.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

It will be appreciated that compounds of the present invention are contemplated as chemically feasible compounds. Accordingly, it will be understood by one of ordinary skill in the art that substituents will satisfy general rules of valency.

Contemplated equivalents of the compounds described above include compounds which otherwise correspond thereto, and which have the same general properties thereof, wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants, which are in themselves known, but are not mentioned here.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this invention. Such compounds are useful, for example, as analytical tools or probes in biological assays.

### DETAILED DESCRIPTION OF CERTAIN EMBODIMENTS

Bi-directional membrane traffic between the Golgi and endosomes plays a vital role in the biogenesis of lysosomes and the localisation of many membrane proteins with diverse physiological functions. The receptors that mediate sorting of lysosomal hydrolases at the Golgi traffic rapidly between the Golgi and endosomes to deliver newly synthesised hydrolases to a pre-lysosomal endosome before returning to the Golgi to repeat the process. The mislocalisation of endosomal and/or lysosomal proteins due to aberrant protein sorting can give rise to a range of pathologies, and there are emerging strands of evidence that defects in the endosome-to-Golgi retrieval pathway contribute significantly to neurodegenerative diseases such as Alzheimer's disease.

The retromer complex that is conserved from yeast to humans plays a major role in endosomal protein sorting and is required for endosome-to-Golgi retrieval. The retromer is a multi-subunit complex that associates with the cytosolic face of endosomes and mediates retrograde transport of transmembrane proteins from endosomes to the trans-Golgi network (TGN).

As stated above, biochemical and genetic studies in yeast and higher eukaryotes have identified two distinct retromer sub-complexes; a core trimer composed of VPS35-VPS29-VPS26 (VPS: vacuolar protein sorting) and an associated homo or heterodimer of sorting nexin (SNX) proteins, containing combinations of SNX1, SNX2, SNX5 and SNX6. The current model postulates that the core complex is a cargo loading assembly that binds to the cytoplasmic tails of trafficking receptors such as the cation independent mannose-6-phosphate receptor (CI-MPR), Wntless, sortilin and DMT1 via the large VPS35 subunit (Arighi C N, Hartnell L M, Aguilar R C, Haft C R, Bonifacino J S (2004) Role of the mammalian retromer in sorting of the cation-independent mannose 6-phosphate receptor. J Cell Biol 165: 123-133; Belenkaya T Y, Wu Y, Tang X, Zhou B, Cheng L, et al. (2008) The retromer complex influences Wnt secretion by recycling wntless from endosomes to the trans-Golgi network. Dev Cell 14: 120-131; Canuel M, Lefrancois S, Zeng J, Morales C R (2008) AP-1 and retromer play opposite roles in the trafficking of sortilin between the Golgi apparatus and the lysosomes. Biochem Biophys Res Commun 366: 724-730; Coudreuse D Y, Roel G, Betist M C, Destree O, Korswagen H C (2006) Wnt gradient formation requires retromer function inWnt-producing cells. Science 312: 921-924; Franch-Marro X, Wendler F, Guidato S, Griffith J, Baena-Lopez A, et al. (2008) Wingless secretion requires endosome-to-Golgi retrieval of Wntless/Evi/Sprinter by the retromer complex. Nat Cell Biol 10: 170-177; Nothwehr S F, Bruinsma P, Strawn L A (1999) Distinct domains within Vps35p mediate the retrieval of two different cargo proteins from the yeast prevacuolar/endosomal compartment. Mol Biol Cell 10: 875-890; Seaman M N (2004) Cargo-selective endosomal sorting for retrieval to the Golgi requires retromer. J Cell Biol 165: 111-122; Seaman M N (2007) Identification of a novel conserved sorting motif required for retromer-mediated endosome-to-TGN retrieval. J Cell Sci 120: 2378-2389; Seaman M N, McCaffery J M, Emr S D (1998) A membrane coat complex essential for endosome-to-Golgi retrograde transport in yeast. J Cell Biol 142: 665-681; Verges M, Luton F, Gruber C, Tiemann F, Reinders L G, et al. (2004) The mammalian retromer regulates transcytosis of the polymeric immunoglobulin receptor. Nat Cell Biol 6: 763-769; Tabuchi M, Yanatori I, Kawai Y, Kishi F (2010) Retromer-mediated direct sorting is required for proper endosomal recycling of the mammalian iron transporter DMT1. J Cell Sci 123: 756-766).

The SNX proteins drive the membrane remodeling required to form the tubulovesicular transport structures (Carlton J, Bujny M, Peter B J, Oorschot V M, Rutherford A, et al. (2004) Sorting Nexin-1 Mediates Tubular Endosome-to-TGN Transport through Coincidence Sensing of High-Curvature Membranes and 3-Phosphoinositides. Curr Biol 14: 1791-1800; Cullen P J (2008) Endosomal sorting and signalling: an emerging role for sorting nexins. Nat Rev Mol Cell Biol 9: 574-582; Nakada-Tsukui K, Saito-Nakano Y, Ali V, Nozaki T (2005) A retromerlike complex is a novel Rab7 effector that is involved in the transport of the virulence factor cysteine protease in the enteric protozoan parasite Entamoeba histolytica. Mol Biol Cell 16: 5294-5303; Rojas R, van Vlijmen T, Mardones G A, Prabhu Y, Rojas A L, et al. (2008) Regulation of retromer recruitment to endosomes by sequential action of Rab5 and Rab7. J Cell Biol 183: 513-526), and along with the small GTPase Rab7 may regulate recruitment of retromer to endosomal membranes through binding to phosphatidylinositol-3-phosphate (PtdIns(3)P).

In some embodiments, a retromer component is the retromer complex protein, VPS35 (GenBank Accession No. BC002414 for human VPS35).

In some embodiments, retromer (the retromer complex) comprises one or more of the following retromer complex proteins: VPS17 (GenBank Accession No. NC00147 for yeast VPS17), VPS26 (GenBank Accession No. BC022505 for human VPS26), VPS29 (GenBank Accession No. BC000880 for human VPS29), sorting nexin 1 (GenBank Accession No. AF065483 for human sorting nexin 1) and sorting nexin 2 (GenBank Accession No. AF065482 for human sorting nexin 2).

Vacuolar protein sorting-associated protein 35 (i.e., VPS35), is a protein that in humans is encoded by the VPS35 gene, which belongs to a group of vacuolar protein sorting (VPS) genes. The wild type VPS35 protein is a 796 amino acid polypeptide (SEQ ID NO: 1) with an approximate molecular weight of ˜92 kDa.

(SEQ ID NO: 1)
MPTTQQSPQD EQEKLLDEAI QAVKVQSFQM KRCLDKNKLM
DALKHASNML GELRTSMLSP KSYYELYMAI SDELHYLEVY
LTDEFAKGRK VADLYELVQY AGNIIPRLYL LITVGVVYVK
SFPQSRKDIL KDLVEMCRGV QHPLRGLFLR NYLLQCTRNI
LPDEGEPTDE ETTGDISDSM DFVLLNFAEM NKLWVRMQHQ
GHSRDREKRE RERQELRILV GTNLVRLSQL EGVNVERYKQ
IVLTGILEQV VNCRDALAQE YLMECIIQVF PDEFHLQTLN
PFLRACAELH QNVNVKNIII ALIDRLALFA HREDGPGIPA
DIKLFDIFSQ QVATVIQSRQ DMPSEDVVSL QVSLINLAMK
CYPDRVDYVD KVLETTVEIF NKLNLEHIAT SSAVSKELTR
LLKIPVDTYN NILTVLKLKH FHPLFEYFDY ESRKSMSCYV
LSNVLDYNTE IVSQDQVDSI MNLVSTLIQD QPDQPVEDPD
PEDFADEQSL VGRFIHLLRS EDPDQQYLIL NTARKHFGAG
GNQRIRFTLP PLVFAAYQLA FRYKENSKVD DKWEKKCQKI
FSFAHQTISA LIKAELAELP LRLFLQGALA AGEIGFENHE
TVAYEFMSQA FSLYEDEISD SKAQLAAITL IIGTFERMKC
FSEENHEPLR TQCALAASKL LKKPDQGRAV STCAHLFWSG
RNTDKNGEEL HGGKRVMECL KKALKIANQC MDPSLQVQLF
IEILNRYIYF YEKENDAVTI QVLNQLIQKI REDLPNLESS
EETEQINKHF HNTLEHLRLR RESPESEGPI YEGLIL

As used herein, the terms “VPS35” “VPS35 protein” “VPS35 polypeptide” and the like refer to a polypeptide having an amino acid sequence as set forth in SEQ ID NO: 1 above, or any variants thereof.

There are known polymorphisms of VPS35, which may, without limitation, include one or more of the following amino acid variants (shown with the position of amino acid residues corresponding to the wild type protein with variants in the single-letter amino acid code): 65 (E/G); 77 (L/S); 83 (D/V/E); 202 (H/N); 305 (R/S); 382 (K/R); 453 (S/F); 557 (C/S); 602 (V/D); 626 (A/V); 755 (P/S/Q); 757 (L/F); 782 (E/G); 42 (A/S); 160 (I/T); 168 (T/P); 526 (R/G); 694 (K/E) and 796 (L/H). A VPS35 polypeptide may include one or more of amino acid variants in any combinations.

According to the invention, in some embodiments, a VPS35 polypeptide contains at least one point mutation. In some embodiments, such a point mutation causes an amino acid substitution. In some embodiments, VPS35 polypeptides useful for the present invention differ from the amino acid sequence of SEQ ID NO: 1 by at least one amino acid, e.g., 1, 2, 3, 4, 5, 6, 7, 8, 9, 10, 11, 12, 13, 14, 15, 16, 17, 18, 19, 20 or more. In some embodiments, VPS35 polypeptides useful for the present invention share at least 80% sequence identity as compared to the amino acid sequence of SEQ ID NO: 1. For example, VPS35 polypeptides useful for the present invention share at least 80%, at least 85%, at least 90%, at least 91%, at least 92%, at least 93%, at least 94%, at least 95%, at least 96%, at least 97%, at least 98%, at least 99%, or greater sequence identity as compared to the amino acid sequence set forth as SEQ ID NO: 1.

VPS35 has been shown to directly interact with VPS29, VPS26A and VPS26B. The interaction with VPS29 is via the C-terminal fragment of the VPS35 protein.

According to the present invention, certain mutations in retromer can affect the stability of the retromer complex. In some embodiments, the stability of the retromer complex is rendered by VPS35-VPS29 interaction.

In some embodiments, at least one mutation contained in the VPS35 polypeptide and/or the VPS29 polypeptide affects the interaction between the polypeptides. For example, such a mutation may be present at or near the interface of the VPS35-VPS29 complex (e.g., VPS35-VPS29 binary complex) such that association between the two polypeptides is altered. In some embodiments, the VPS35-VPS29 interface involves the metal binding domain of the VPS29 polypeptide. In some embodiments, the VPS35-VPS29 interface involves one or more of the following amino acid residues on VPS29: positions 8, 10, 14, 39, 62, 63, 86, 91 and 115 (position numbers corresponding to wild type VPS29). In some embodiments, the VPS35-VPS29 interface involves one or more of the following secondary structures of VPS35: alpha helix 1 (α1), alpha helix 3 (α3), alpha helix 5 (α5), alpha helix 7 (α7), alpha helix 9 (α9), alpha helix 11 (α11), and alpha helix 13 (α13). In some embodiments, the VPS35-VPS29 interface involves one or more of the following amino acid residues on VPS35: positions 534, 541, 579, 582, 586, 589, 629, 630, 633, 637, 672, 675, 725, 729, 769, 772 and 776.

In some embodiments, retromer stability is affected by an altered rate of association between or amongst retromer components (e.g., complex formation). Alternatively or additionally, in some embodiments, retromer stability is affected by an altered rate of dissociation of the complex. In some embodiments, a mutation or mutations that affect retromer stability may reduce overall expression levels of retromer components in cells.

In some embodiments, the stability of retromer is determined (e.g., measured) by thermal stability of the retromer complex. Changes in stability may be therefore measured by assaying for thermal stability of retromer complex, for example, in the presence or absence of a retromer stabilizing agent provided herein.

In some embodiments, one or more mutations of a VPS35 polypeptide occur at or near the interface of a VPS35-VPS29 binary complex, e.g., in the C-terminus portion of VPS35. In some embodiments, one or more mutations of a VPS35 polypeptide occur in amino acid residues between ˜520 and ˜780. In some embodiments, such mutations may occur at amino acid residue(s): 316, 534, 524, 541, 579, 582, 586, 589, 620, 629, 630, 633, 637, 672, 675, 725, 729, 769, 772, 776 or any combinations thereof.

In some embodiments, a VPS35 polypeptide has a deletion. Accordingly in some embodiments, a VPS35 polypeptide contains fewer than 796 amino acids.

The Vps26-Vps29-Vps35 trimer is thought to participate in cargo binding and is therefore referred to as the “cargo recognition complex” (for review, see, for example, Bonifacino and Hurley (2008), Curr Opin Cell Biol. 20(4): 427-36, which is incorporated herein by reference). The structure of Vps35 and the structural basis for its interactions with Vps29 has been revealed. A low-resolution molecular structure for the entire Vps26-Vps29-Vps35 cargo recognition complex was developed, and a model for the docking of retromer to tubular endosomes was proposed (Hierro A, Rojas A L, Rojas R, Murthy N, Effantin G, Kajava A V, Steven A C, Bonifacino J S, Hurley J H. Functional architecture of the retromer cargo-recognition complex. Nature 2007; 449:1063-1067).

The crystal structures of human, murine, and Cryptosporidium parvum Vps29 reveal that this subunit has a metallophosphoesterase fold (Hierro A, Rojas A L, Rojas R, Murthy N, Effantin G, Kajava A V, Steven A C, Bonifacino J S, Hurley J H. Functional architecture of the retromer cargo-recognition complex. Nature 2007; 449:1063-1067; Wang D, Guo M, Liang Z, Fan J, Zhu Z, Zang J, Li X, Teng M, Niu L, Dong Y, et al. Crystal structure of human vacuolar protein sorting protein 29 reveals a phosphodiesterase/nuclease-like fold and two protein-protein interaction sites. J Biol Chem 2005; 280:22962-22967; Collins B M, Skinner C F, Watson P J, Seaman M N, Owen D J. Vps29 has a phosphoesterase fold that acts as a protein interaction scaffold for retromer assembly. Nat Struct Mol Biol 2005; 12:594-602). The direct binding of divalent metal ions to the metallophosphoesterase-like active site has been confirmed (Bonifacino and Hurley, Curr Opin Cell Biol. Author manuscript; available in PMC 2010 March 6. NIH-PA Author Manuscript NIH-PA Author Manuscript NIH-PA Author Manuscript structurally for murine Vps29). Active metallophosphoesterases contain a His residue that serves as a catalytic base and is required for activity. Vps29 contains instead a Phe residue at this position, consistent with the absence of catalytic activity. Based on the structure of the Vps29-Vps35 subcomplex and on mutational analyses available in literature, the metalbinding face of Vps29 may serve as a scaffold for the assembly of Vps35. Mutational studies also show that Vps29 contains a SNX binding site (Collins B M, Norwood S J, Kerr M C, Mahony D, Seaman M N, Teasdale R D, Owen D J. Structure of Vps26B and mapping of its interaction with the retromer protein complex. Traffic. 2007), which is on the opposite face of the protein from the Vps35 binding site.

The C-terminal ˜40% of the human VPS35 has been crystallized in complex with VPS29 (e.g., a “VPS35/VPS29 binary complex”), revealing an α-solenoid fold that curves around the metal-binding face of Vps29 (Hierro et al., supra). The crystal structure studies indicate that the C-terminal portion of VPS35 consists of a single right-handed superhelix with a pitch of 12 Å and a total of 13 helices. VPS35 resembles many other helical solenoid proteins. VPS35 has been shown to wrap itself nearly halfway around the VPW29 subunit, burying 3300 Å² of solvent-accessible surface area. The VPS35 binding site on VPS29 includes the entire metal-binding site, as well as flanking residues. At or near the Ile91 residue of VPS29, which was previously shown to contact VPS35, interacts extensively with VPS35 near the center of the interface of the VPS35-VPS29 binary complex. Bioinformatics analysis guided by the structure shows that the α-solenoid extends through the entire structure of VPS35.

Accordingly, in some embodiments, a mutation or mutations that affect the interaction between VPS35 and VPS29 at least in part determines the stability of the retromer complex. As provided in further detail herein, the present invention provides retromer stabilizing agents that bind to retromer (whole complex or sub-complex) to stabilize the complex. Thus, the present invention encompasses the idea that retromer stabilizing agents may be used to overcome destabilized cellular retromer due to one or more mutations present in at least one retromer component. Accordingly, the invention provides compositions and related methods for “correcting” destabilized cellular retromer by the use of retromer stabilizing agents so as to restore functional retromer-mediated protein sorting machinery in cells. In some embodiments of the invention, retromer-stabilizing agents provided herein can accelerate (e.g., further facilitate) the activity of cellular retromer in protein trafficking. In some embodiments, retromer-stabilizing agents provided herein can restore normal function of cellular retromer, which, in the absence of such agents, has impaired function.

Thus, the present invention provides retromer stabilizing agents as further described below.

In some embodiments, the invention provides a compound of formula I:

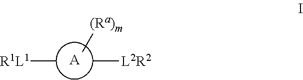

or a pharmaceutically acceptable salt thereof, wherein Ring A, R^{a}, m, L¹, L², R¹, and R², are as defined herein, for use in the treatment of neurodegenerative diseases featuring amyloid. The present invention also provides methods of preparing such compounds and various compositions and uses of such compounds.

In some embodiments, the present invention provides methods of treating a subject suffering from or susceptible to a neurodegenerative disease, disorder or condition with a compound of Formula I. In certain embodiments, the subject is an adult human. In certain embodiments, the neurodegenerative disease, disorder or condition is a disease, disorder, or conditions featuring amyloids. Exemplary such diseases, disorders, or conditions may include, but are not limited to, Alzheimer's Disease, Diabetes mellitus type 2, Parkinson's Disease, Transmissible spongiform encephalopathy (e.g., bovine spongiform encephalopathy), Huntington's Disease, medullary carcinoma of the thyroid, cardiac arrythmias, isolated atrial amyloidosis, atherosclerosis, rheumatoid arthritis, aortic medial amyloid, prolactinomas, familial amyloid polyneuropathy, hereditary non-neuropathic systemic amyloidosis, dialysis related amyloidosis, Finnish amyloidosis, lattice corneal dystrophy, cerebral amyloid angiopathy, cerebral amyloid angiopathy (Icelandic type), systemic AL amyloidosis, sporadic inclusion body mytosis, diffuse Lewy Body Disease, multiple system atrophy (MSA), cortico basal degeneration (CBD), pgrogressive supranuclear palsy (PSP), Lewy Body Disease/Lewy Body Dementia/Dementia with Lewy Bodies, pantothenate kinase-associated neurodegeneration (PANK1), and amyotrophic lateral sclerosis (ALS).

All publications and patent documents cited in this application are incorporated herein by reference in their entirety.

### General Description of Compounds of the Invention

In some embodiments, the present invention provides a compound of formula I:

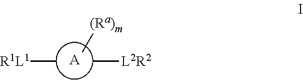

or a pharmaceutically acceptable salt thereof, wherein:
Ring A is an optionally substituted bivalent 3-8 membered saturated, partially unsaturated, or aryl monocyclic ring having 0-4 heteroatoms independently selected from nitrogen, oxygen, or sulfur, or an optionally substituted bivalent 8-10 membered saturated, partially unsaturated, or aryl bicyclic ring having 0-4 heteroatoms independently selected from nitrogen, oxygen, or sulfur;
m is 0-5;
each R^{a} is independently —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂;
each R is independently deuterium, hydrogen, halogen, an optionally substituted C₁₋₆ aliphatic group, or an optionally substituted 3-8 membered saturated, partially unsaturated, or aryl ring having 0-4 heteroatoms independently selected from nitrogen, oxygen, or sulfur, or wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur, or wherein:
two R on the same carbon are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur; and
L¹ and L² are each independently a valence bond or a bivalent optionally substituted C₁₋₁₀ alkylene chain wherein one, two, or three methylene units are optionally and independently replaced by —O—, —N(R)—, —S—, —C(O)—, —C(═NR)— —OC(O)—, —C(O)O—, —OC(O)O—, —S(O)—, —S(O)₂—, —OSO₂O—, —N(R)C(O)—, —C(O)N(R)—, —N(R)C(O)O—, —OC(O)NR—, —N(R)C(O)NR—, and wherein L¹ and L² are each independently optionally substituted with 1-6 R groups; and
R¹ and R² are each independently selected from —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂, —SC(═NR)N(R)₂, or an optionally substituted C₁₋₂₀ aliphatic group.

In some embodiments, a compound of formula I is a salt.

In some embodiments, a compound of formula I is a pharmaceutically acceptable salt.

Exemplary salts include, but are not limited to, those salts described herein in the section entitled “Uses of Compounds and Pharmaceutically Acceptable Compositions.” In some embodiments, a compound of formula I is in the form of a dihydrohalide salt. In certain embodiments, a compound of formula I is in the form of a dihydrochloride salt.

As described generally above and defined herein, Ring A is an optionally substituted bivalent 3-8 membered saturated, partially unsaturated, or aryl monocyclic ring having 0-4 heteroatoms independently selected from nitrogen, oxygen, or sulfur, or an optionally substituted bivalent 8-10 membered saturated, partially unsaturated, or aryl bicyclic ring having 0-4 heteroatoms independently selected from nitrogen, oxygen, or sulfur.

In some embodiments, Ring A is a bivalent optionally substituted saturated monocyclic ring. In some embodiments, Ring A is a bivalent optionally substituted partially unsaturated monocyclic ring. In some embodiments, Ring A is a bivalent optionally substituted aromatic monocyclic ring.

In some embodiments, Ring A is a bivalent optionally substituted saturated bicyclic ring. In some embodiments, Ring A is a bivalent optionally substituted partially unsaturated bicyclic ring. In some embodiments, Ring A is a bivalent optionally substituted aromatic bicyclic ring.

In some embodiments, Ring A is an optionally substituted 6-10 membered arylene. In some embodiments, Ring A is an optionally substituted a 5-10 membered heteroarylene having 1-4 heteroatoms independently selected from oxygen, nitrogen, or sulfur. In some embodiments, Ring A is an optionally substituted a 5-6 membered heteroarylene having 1-4 heteroatoms independently selected from oxygen, nitrogen, or sulfur. In some embodiments, Ring A is an optionally substituted 5 membered heteroarylene having 1-4 heteroatoms independently selected from oxygen, nitrogen, or sulfur. In some embodiments, Ring A is an optionally substituted 6 membered heteroarylene having 1-4 heteroatoms independently selected from oxygen, nitrogen, or sulfur.

Exemplary optionally substituted Ring A heteroarylene groups include thienylene, furanylene, pyrrolylene, imidazolylene, pyrazolylene, triazolylene, tetrazolylene, oxazolylene, isoxazolylene, oxadiazolylene, thiazolylene, isothiazolylene, thiadiazolylene, pyridylene, pyridazinylene, pyrimidinylene, pyrazinylene, indolizinylene, purinylene, naphthyridinylene, pteridinylene, indolylene, isoindolylene, benzothienylene, benzofuranylene, dibenzofuranylene, indazolylene, benzimidazolylene, benzthiazolylene, quinolylene, isoquinolylene, cinnolinylene, phthalazinylene, quinazolinylene, quinoxalinylene, 4H-quinolizinylene, carbazolylene, acridinylene, phenazinylene, phenothiazinylene, phenoxazinylene, tetrahydroquinolinylene, tetrahydroisoquinolinylene, pyrido[2,3-b]-1,4-oxazin-3(4H)-onylene, and chromanylene.

In certain embodiments, Ring A is optionally substituted thienylene.

In certain embodiments, Ring A is optionally substituted furanylene.

In certain embodiments, Ring A is optionally substituted pyrrolylene.

In certain embodiments, Ring A is optionally substituted phenylene.

In some embodiments, Ring A is an optionally substituted 3-8 membered carbocyclylene. In some embodiments, Ring A is an optionally substituted 3-6 membered carbocyclylene.

In some embodiments, Ring A is an optionally substituted 3-10 membered heterocyclylene having 1-4 heteroatoms independently selected from oxygen, nitrogen, or sulfur. In some embodiments, Ring A is an optionally substituted 5-7 membered heterocyclylene having 1-3 heteroatoms independently selected from oxygen, nitrogen, or sulfur. In some embodiments, Ring A is an optionally substituted 3 membered heterocyclylene having 1 heteroatom independently selected from oxygen, nitrogen, or sulfur. In some embodiments, Ring A is an optionally substituted 5 membered heterocyclylene having 1-2 heteroatoms independently selected from oxygen, nitrogen, or sulfur. In some embodiments, Ring A is an optionally substituted 6 membered heterocyclylene having 1-3 heteroatoms independently selected from oxygen, nitrogen, or sulfur.

In some embodiments, Ring A is an optionally substituted partially unsaturated 4-10 membered heterocyclylene having 1-4 heteroatoms independently selected from oxygen, nitrogen, or sulfur. In some embodiments, Ring A is an optionally substituted partially unsaturated 5-7 membered heterocyclylene having 1-3 heteroatoms independently selected from oxygen, nitrogen, or sulfur. In some embodiments, Ring A is an optionally substituted partially unsaturated 5 membered heterocyclylene having 1-2 heteroatoms independently selected from oxygen, nitrogen, or sulfur. In some embodiments, Ring A is an optionally substituted partially unsaturated 6 membered heterocyclylene having 1-3 heteroatoms independently selected from oxygen, nitrogen, or sulfur.

Exemplary Ring A partially unsaturated 5 membered optionally substituted heterocyclylenes include dihydrothienylene, dihydropyrrolylene, dihydroimidazolylene, dihydrooxazolylene, dihydrothiazolylene, dihydrothiadiazolylene, and dihydrooxadiazolylene.

Exemplary Ring A saturated 3-8 membered optionally substituted heterocyclylenes include oxiranylene, oxetanylene, tetrahydrofuranylene, tetrahydropyranylene, oxepaneylene, aziridineylene, azetidineylene, pyrrolidinylene, piperidinylene, azepanylene, thiiranylene, thietanylene, tetrahydrothiophenylene, tetrahydrothiopyranylene, thiepanylene, dioxolanylene, oxathiolanylene, oxazolidinylene, imidazolidinylene, thiazolidinylene, dithiolanylene, dioxanylene, morpholinylene, oxathianylene, piperazinylene, thiomorpholinylene, dithianylene, dioxepanylene, oxazepanylene, oxathiepanylene, dithiepanylene, diazepanylene, dihydrofuranonylene, tetrahydropyranonylene, oxepanonylene, pyrolidinonylene, piperidinonylene, azepanonylene, dihydrothiophenonylene, tetrahydrothiopyranonylene, thiepanonylene, oxazolidinonylene, oxazinanonylene, oxazepanonylene, dioxolanonylene, dioxanonylene, dioxepanonylene, oxathiolinonylene, oxathianonylene, oxathiepanonylene, thiazolidinonylene, thiazinanonylene, thiazepanonylene, imidazolidinonylene, tetrahydropyrimidinonylene, diazepanonylene, imidazolidinedionylene, oxazolidinedionylene, thiazolidinedionylene, dioxolanedionylene, oxathiolanedionylene, piperazinedionylene, morpholinedionylene, and thiomorpholinedionylene.

In some embodiments, Ring A is of any one of the formulae:

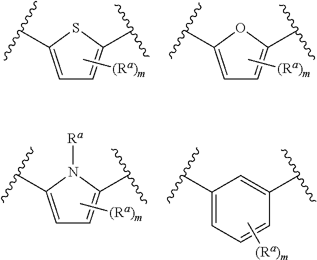

wherein each of R^{a} and m are as defined and described herein.

In some embodiments, Ring A is of any one of the formulae:

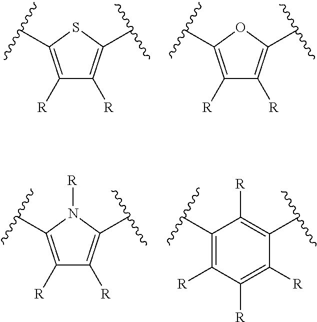

wherein each R is as defined and described herein.

In some embodiments, Ring A is of any one of the formulae:

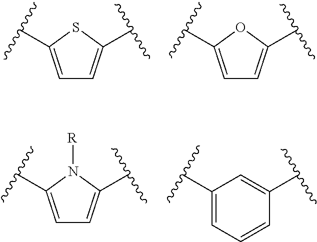

wherein R is as defined and described herein.

As described generally above and defined herein, L¹ and L² are each independently a valence bond or a bivalent optionally substituted C₁₋₁₀ alkylene chain wherein one, two, or three methylene units are optionally and independently replaced by —O—, —N(R)—, —S—, —C(O)—, —C(═NR)— —OC(O)—, —C(O)O—, —OC(O)O—, —S(O)—, —S(O)₂—, —OSO₂O—, —N(R)C(O)—, —C(O)N(R)—, —N(R)C(O)O—, —OC(O)NR—, or —N(R)C(O)NR—, and wherein L¹ and L² are each independently optionally substituted with 1-6 R groups.

In some embodiments, L¹ and L² are the same.

In some embodiments, L¹ and L² are different.

In some embodiments, L¹ is a valence bond.

In some embodiments, L¹ is an optionally substituted C₁₋₁₀ alkylene chain wherein one, two, or three methylene units are independently replaced by —O—, —N(R)—, —S—, —C(O)—, —C(═NR)—, —OC(O)—, —C(O)O—, —OC(O)O—, —S(O)—, —S(O)₂—, —OSO₂O—, —NRC(O)—, —C(O)NR—, —N(R)C(O)O—, —OC(O)NR—, or —N(R)C(O)NR—.

In some embodiments L¹ is a C₁ alkylene chain optionally substituted with 1-6 R groups. In some embodiments L¹ is a C₂ alkylene chain optionally substituted with 1-6 R groups. In some embodiments L¹ is a C₃ alkylene chain optionally substituted with 1-6 R groups. In some embodiments L¹ is a C₄ alkylene chain optionally substituted with 1-6 R groups. In some embodiments L¹ is a C₅ alkylene chain optionally substituted with 1-6 R groups. In some embodiments L¹ is a C₆ alkylene chain optionally substituted with 1-6 R groups. In some embodiments L¹ is a C₇ alkylene chain optionally substituted with 1-6 R groups. In some embodiments L¹ is a C₈ alkylene chain optionally substituted with 1-6 R groups. In some embodiments L¹ is a C₉ alkylene chain optionally substituted with 1-6 R groups. In some embodiments L¹ is a C₁₀ alkylene chain optionally substituted with 1-6 R groups. In certain embodiments, one or more of the 1-6 R groups are a halogen. In certain embodiments, one or more of the 1-6 R groups are fluorine.

In certain embodiments, L¹ is an optionally substituted C₁₋₁₀ alkylene chain wherein one, two, or three methylene units are independently replaced by —O—, —N(R)—, —S—, —C(O)—, or —C(═NR)—. In certain embodiments, L¹ is an optionally substituted C₂₋₁₀ alkylene chain wherein two or more methylene units are independently replaced by —N(R)—, —S—, —C(O)—, or —C(═NR)—. In certain embodiments, L¹ is an optionally substituted C₂₋₁₀ alkylene chain wherein at least one methylene unit is replaced by —N(R)—. In certain embodiments, L¹ is an optionally substituted C₂₋₁₀ alkylene chain wherein at least one methylene unit is replaced by —C(═NR)—. In certain embodiments, L¹ is an optionally substituted C₂₋₁₀ alkylene chain wherein at least one methylene unit is replaced by —S—. In certain embodiments, L¹ is an optionally substituted C₃₋₁₀ alkylene chain wherein three methylene units are independently replaced by —S—, —C(═NR)—, and —N(R)—. In certain embodiments, L¹ is an optionally substituted C₃₋₁₀ alkylene chain wherein three adjacent methylene units are independently replaced by —S—, —C(═NR)—, and —N(R)—. In certain embodiments, L¹ is an optionally substituted C₂₋₁₀ alkylene chain wherein two adjacent methylene units are independently replaced by —N(R)— and —C(═NR)—. In certain embodiments, L¹ is an optionally substituted C₂₋₁₀ alkylene chain wherein two adjacent methylene units are independently replaced by —S— and —C(═NR)—.

In certain embodiments, L¹ is an optionally substituted C₄₋₆ alkylene chain wherein one, two, or three methylene units are independently replaced by —O—, —N(R)—, —S—, —C(O)—, —C(═NR)—, —OC(O)—, —C(O)O—, —OC(O)O—, —S(O)—, —S(O)₂—, —OSO₂O—, —NRC(O)—, —C(O)NR—, —N(R)C(O)O—, —OC(O)NR—, or —N(R)C(O)NR—. In certain embodiments, L¹ is an optionally substituted C₄₋₆ alkylene chain wherein one, two, or three methylene units are independently replaced by —O—, —N(R)—, —S—, —C(O)—, or —C(═NR)—. In certain embodiments, L¹ is an optionally substituted C₄ alkylene chain wherein three methylene units are independently replaced by —N(R)—, —S—, or —C(═NR)—.

In some embodiments, L² is a valence bond.

In some embodiments, L² is an optionally substituted C₁₋₁₀ alkylene chain wherein one, two, or three methylene units are independently replaced by —O—, —N(R)—, —S—, —C(O)—, —C(═NR)—, —OC(O)—, —C(O)O—, —OC(O)O—, —S(O)—, —S(O)₂—, —OSO₂O—, —NRC(O)—, —C(O)NR—, —N(R)C(O)O—, —OC(O)NR—, or —N(R)C(O)NR—.

In some embodiments L² is a C₁ alkylene chain optionally substituted with 1-6 R groups. In some embodiments L² is a C₂ alkylene chain optionally substituted with 1-6 R groups. In some embodiments L² is a C₃ alkylene chain optionally substituted with 1-6 R groups. In some embodiments L² is a C₄ alkylene chain optionally substituted with 1-6 R groups. In some embodiments L² is a C₅ alkylene chain optionally substituted with 1-6 R groups. In some embodiments L² is a C₆ alkylene chain optionally substituted with 1-6 R groups. In some embodiments L² is a C₇ alkylene chain optionally substituted with 1-6 R groups. In some embodiments L² is a C₈ alkylene chain optionally substituted with 1-6 R groups. In some embodiments L² is a C₉ alkylene chain optionally substituted with 1-6 R groups. In some embodiments L² is a C₁₀ alkylene chain optionally substituted with 1-6 R groups. In certain embodiments, one or more of the 1-6 R groups are a halogen. In certain embodiments, one or more of the 1-6 R groups are fluorine.

In certain embodiments, L² is an optionally substituted C₁₋₁₀ alkylene chain wherein one, two, or three methylene units are independently replaced by —O—, —N(R)—, —S—, —C(O)—, or —C(═NR)—. In certain embodiments, L² is an optionally substituted C₂₋₁₀ alkylene chain wherein two or more methylene units are independently replaced by —N(R)—, —S—, —C(O)—, or —C(═NR)—. In certain embodiments, L² is an optionally substituted C₂₋₁₀ alkylene chain wherein at least one methylene unit is replaced by —N(R)—. In certain embodiments, L² is an optionally substituted C₂₋₁₀ alkylene chain wherein at least one methylene unit is replaced by —C(═NR)—. In certain embodiments, L² is an optionally substituted C₂₋₁₀ alkylene chain wherein at least one methylene unit is replaced by —S—. In certain embodiments, L² is an optionally substituted C₃₋₁₀ alkylene chain wherein three methylene units are independently replaced by —S—, —C(═NR)—, and —N(R)—. In certain embodiments, L² is an optionally substituted C₃₋₁₀ alkylene chain wherein three adjacent methylene units are independently replaced by —S—, —C(═NR)—, and —N(R)—. In certain embodiments, L² is an optionally substituted C₂₋₁₀ alkylene chain wherein two adjacent methylene units are independently replaced by —N(R)— and —C(═NR)—. In certain embodiments, L² is an optionally substituted C₂₋₁₀ alkylene chain wherein two adjacent methylene units are independently replaced by —S— and —C(═NR)—.

In certain embodiments, L² is an optionally substituted C₄₋₆ alkylene chain wherein one, two, or three methylene units are independently replaced by —O—, —N(R)—, —S—, —C(O)—, —C(═NR)—, —OC(O)—, —C(O)O—, —OC(O)O—, —S(O)—, —S(O)₂—, —OSO₂O—, —NRC(O)—, —C(O)NR—, —N(R)C(O)O—, —OC(O)NR—, or —N(R)C(O)NR—. In certain embodiments, L² is an optionally substituted C₄₋₆ alkylene chain wherein one, two, or three methylene units are independently replaced by —O—, —N(R)—, —S—, —C(O)—, or —C(═NR)—. In certain embodiments, L² is an optionally substituted C₄ alkylene chain wherein three methylene units are independently replaced by —N(R)—, —S—, or —C(═NR)—.

As described generally above and defined herein, R¹ and R² are each independently selected from —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂, —SC(═NR)N(R)₂, or an optionally substituted C₁₋₂₀ aliphatic group.

In some embodiments, R¹ and R² are the same. In some embodiments, R¹ and R² are different.

In some embodiments, R¹ is R. In some embodiments, R¹ is hydrogen.

In some embodiments, R¹ is an optionally substituted C₁₋₂₀ aliphatic group. In some embodiments, R¹ is an optionally substituted C₁₋₁₉ aliphatic group. In some embodiments, R¹ is an optionally substituted C₁₋₁₈ aliphatic group. In some embodiments, R¹ is an optionally substituted C₁₋₁₇ aliphatic group. In some embodiments, R¹ is an optionally substituted C₁₋₁₆ aliphatic group. In some embodiments, R¹ is an optionally substituted C₁₋₁₅ aliphatic group. In some embodiments, R¹ is an optionally substituted C₁₋₁₄ aliphatic group. In some embodiments, R¹ is an optionally substituted C₁₋₁₃ aliphatic group. In some embodiments, R¹ is an optionally substituted C₁₋₁₂ aliphatic group. In some embodiments, R¹ is an optionally substituted C₁₋₁₁ aliphatic group. In some embodiments, R¹ is an optionally substituted C₁₋₁₀ aliphatic group. In some embodiments, R¹ is an optionally substituted C₁₋₉ aliphatic group. In some embodiments, R¹ is an optionally substituted C₁₋₉ aliphatic group. In some embodiments, R¹ is an optionally substituted C₁₋₇ aliphatic group. In some embodiments, R¹ is an optionally substituted C₁₋₆ aliphatic group. In some embodiments, R¹ is an optionally substituted C₆ aliphatic group. In some embodiments, R¹ is an optionally substituted C₅ aliphatic group. In some embodiments, R¹ is an optionally substituted C₄ aliphatic group. In some embodiments, R¹ is an optionally substituted C₃ aliphatic group. In some embodiments, R¹ is an optionally substituted C₂ aliphatic group. In some embodiments, R¹ is an optionally substituted C₁ aliphatic group.

In certain embodiments, R¹ is an optionally substituted 3-8 membered saturated monocyclic ring having 1-3 heteroatoms independently selected from nitrogen, oxygen, or sulfur. In certain embodiments, R¹ is an optionally substituted 3-8 membered saturated monocyclic carbocycle. In certain embodiments, R¹ is an optionally substituted 5-6 membered saturated monocyclic ring having 1-2 heteroatoms independently selected from nitrogen, oxygen, or sulfur. In certain embodiments, R¹ is an optionally substituted 5-6 membered saturated monocyclic carbocycle. In certain embodiments, R¹ is an optionally substituted 7 membered saturated monocyclic ring having 1-2 heteroatoms independently selected from nitrogen, oxygen, or sulfur. In certain embodiments, R¹ is an optionally substituted 7 membered saturated monocyclic carbocycle.

Exemplary R¹ saturated 3-8 membered optionally substituted heterocycles include oxirane, oxetane, tetrahydrofuran, tetrahydropyran, oxepane, aziridine, azetidine, pyrrolidine, piperidine, azepane, thiirane, thietane, tetrahydrothiophene, tetrahydrothiopyran, thiepane, dioxolane, oxathiolane, oxazolidine, imidazolidine, thiazolidine, dithiolane, dioxane, morpholine, oxathiane, piperazine, thiomorpholine, dithiane, dioxepane, oxazepane, oxathiepane, dithiepane, diazepane, dihydrofuranone, tetrahydropyranone, oxepanone, pyrolidinone, piperidinone, azepanone, dihydrothiophenone, tetrahydrothiopyranone, thiepanone, oxazolidinone, oxazinanone, oxazepanone, dioxolanone, dioxanone, dioxepanone, oxathiolinone, oxathianone, oxathiepanone, thiazolidinone, thiazinanone, thiazepanone, imidazolidinone, tetrahydropyrimidinone, diazepanone, imidazolidinedione, oxazolidinedione, thiazolidinedione, dioxolanedione, oxathiolanedione, piperazinedione, morpholinedione, and thiomorpholinedione.

In certain embodiments, R¹ is an optionally substituted 3-8 membered partially unsaturated monocyclic ring having 1-3 heteroatoms independently selected from nitrogen, oxygen, or sulfur. In certain embodiments, R¹ is an optionally substituted 3-8 membered partially unsaturated monocyclic carbocycle. In certain embodiments, R¹ is an optionally substituted 5-6 membered partially unsaturated monocyclic ring having 1-2 heteroatoms independently selected from nitrogen, oxygen, or sulfur. In certain embodiments, R¹ is an optionally substituted 5-6 membered partially unsaturated monocyclic carbocycle. In certain embodiments, R¹ is an optionally substituted 5-6 membered aryl ring having 0-3 heteroatoms independently selected from nitrogen, oxygen, or sulfur. In certain embodiments, R¹ is an optionally substituted 5 membered aryl ring having 1-3 heteroatoms independently selected from nitrogen, oxygen, or sulfur. In certain embodiments, R¹ is an optionally substituted 6 membered aryl ring having 1-3 heteroatoms independently selected from nitrogen, oxygen, or sulfur. In certain embodiments, R¹ is an optionally substituted phenyl.

In some embodiments, R² is R. In some embodiments, R² is hydrogen.

In some embodiments, R¹ is an optionally substituted C₁₋₂₀ aliphatic group. In some embodiments, R² is an optionally substituted C₁₋₁₉ aliphatic group. In some embodiments, R² is an optionally substituted C₁₋₁₈ aliphatic group. In some embodiments, R² is an optionally substituted C₁₋₁₇ aliphatic group. In some embodiments, R² is an optionally substituted C₁₋₁₆ aliphatic group. In some embodiments, R² is an optionally substituted C₁₋₁₅ aliphatic group. In some embodiments, R² is an optionally substituted C₁₋₁₄ aliphatic group. In some embodiments, R² is an optionally substituted C₁₋₁₃ aliphatic group. In some embodiments, R² is an optionally substituted C₁₋₁₂ aliphatic group. In some embodiments, R² is an optionally substituted C₁₋₁₁ aliphatic group. In some embodiments, R² is an optionally substituted C₁₋₁₀ aliphatic group. In some embodiments, R² is an optionally substituted C₁₋₉ aliphatic group. In some embodiments, R² is an optionally substituted C₁₋₈ aliphatic group. In some embodiments, R² is an optionally substituted C₁₋₇ aliphatic group. In some embodiments, R² is an optionally substituted C₁₋₆ aliphatic group. In some embodiments, R² is an optionally substituted C₆ aliphatic group. In some embodiments, R² is an optionally substituted C₅ aliphatic group. In some embodiments, R² is an optionally substituted C₄ aliphatic group. In some embodiments, R² is an optionally substituted C₃ aliphatic group. In some embodiments, R² is an optionally substituted C₂ aliphatic group. In some embodiments, R² is an optionally substituted C₁ aliphatic group.

In certain embodiments, R² is an optionally substituted 3-8 membered saturated monocyclic ring having 1-3 heteroatoms independently selected from nitrogen, oxygen, or sulfur. In certain embodiments, R² is an optionally substituted 3-8 membered saturated monocyclic carbocycle. In certain embodiments, R² is an optionally substituted 5-6 membered saturated monocyclic ring having 1-2 heteroatoms independently selected from nitrogen, oxygen, or sulfur. In certain embodiments, R² is an optionally substituted 5-6 membered saturated monocyclic carbocycle. In certain embodiments, R² is an optionally substituted 7 membered saturated monocyclic ring having 1-2 heteroatoms independently selected from nitrogen, oxygen, or sulfur. In certain embodiments, R² is an optionally substituted 7 membered saturated monocyclic carbocycle.

Exemplary R² saturated 3-8 membered optionally substituted heterocycles include oxirane, oxetane, tetrahydrofuran, tetrahydropyran, oxepane, aziridine, azetidine, pyrrolidine, piperidine, azepane, thiirane, thietane, tetrahydrothiophene, tetrahydrothiopyran, thiepane, dioxolane, oxathiolane, oxazolidine, imidazolidine, thiazolidine, dithiolane, dioxane, morpholine, oxathiane, piperazine, thiomorpholine, dithiane, dioxepane, oxazepane, oxathiepane, dithiepane, diazepane, dihydrofuranone, tetrahydropyranone, oxepanone, pyrolidinone, piperidinone, azepanone, dihydrothiophenone, tetrahydrothiopyranone, thiepanone, oxazolidinone, oxazinanone, oxazepanone, dioxolanone, dioxanone, dioxepanone, oxathiolinone, oxathianone, oxathiepanone, thiazolidinone, thiazinanone, thiazepanone, imidazolidinone, tetrahydropyrimidinone, diazepanone, imidazolidinedione, oxazolidinedione, thiazolidinedione, dioxolanedione, oxathiolanedione, piperazinedione, morpholinedione, and thiomorpholinedione.

In certain embodiments, R² is an optionally substituted 3-8 membered partially unsaturated monocyclic ring having 1-3 heteroatoms independently selected from nitrogen, oxygen, or sulfur. In certain embodiments, R² is an optionally substituted 3-8 membered partially unsaturated monocyclic carbocycle. In certain embodiments, R² is an optionally substituted 5-6 membered partially unsaturated monocyclic ring having 1-2 heteroatoms independently selected from nitrogen, oxygen, or sulfur. In certain embodiments, R² is an optionally substituted 5-6 membered partially unsaturated monocyclic carbocycle. In certain embodiments, R² is an optionally substituted 5-6 membered aryl ring having 0-3 heteroatoms independently selected from nitrogen, oxygen, or sulfur. In certain embodiments, R² is an optionally substituted 5 membered aryl ring having 1-3 heteroatoms independently selected from nitrogen, oxygen, or sulfur. In certain embodiments, R² is an optionally substituted 6 membered aryl ring having 1-3 heteroatoms independently selected from nitrogen, oxygen, or sulfur. In certain embodiments, R² is an optionally substituted phenyl.

In some embodiments, a compound of Formula I is of either of the formulae:

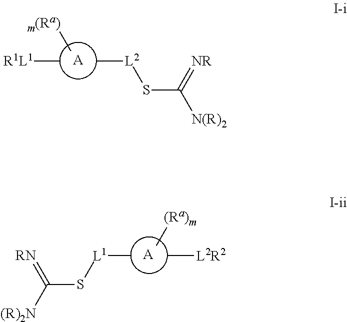

or a pharmaceutically acceptable salt thereof, wherein each of Ring A, R^{a}, m, R¹, L¹, R², L², and R are as defined and described herein.

In some embodiments, a compound of Formula I is of either of the formulae:

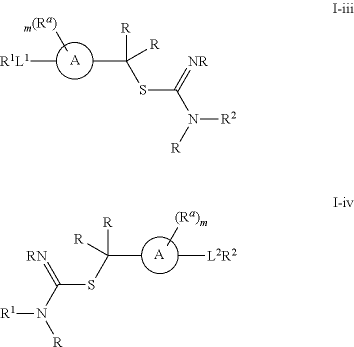

or a pharmaceutically acceptable salt thereof, wherein each of Ring A, R^{a}, m, R¹, L¹, R², L², and R are as defined and described herein.

In some embodiments, a compound of Formula I is of either of the formulae:

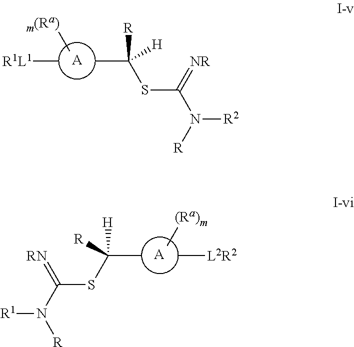

or a pharmaceutically acceptable salt thereof, wherein each of Ring A, R^{a}, m, R¹, L¹, R², L², and R are as defined and described herein.

In some embodiments, a compound of Formula I is of either of the formulae:

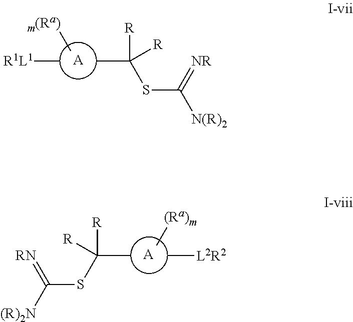

or a pharmaceutically acceptable salt thereof, wherein each of Ring A, R^{a}, m, R¹, L¹, R², L², and R are as defined and described herein.

In some embodiments, a compound of Formula I is of the formula:

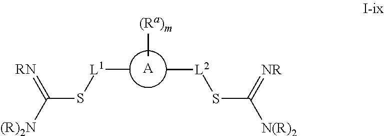

or a pharmaceutically acceptable salt thereof, wherein each of Ring A, R^{a}, m, L¹, L², and R are as defined and described herein.

In some embodiments, a compound of Formula I is of the formula:

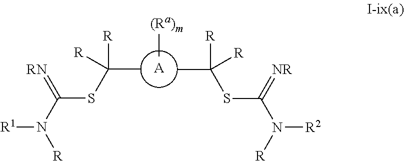

or a pharmaceutically acceptable salt thereof, wherein each of Ring A, R^{a}, m, R¹, R² and R are as defined and described herein.

In some embodiments, a compound of Formula I is of any one of the formulae:

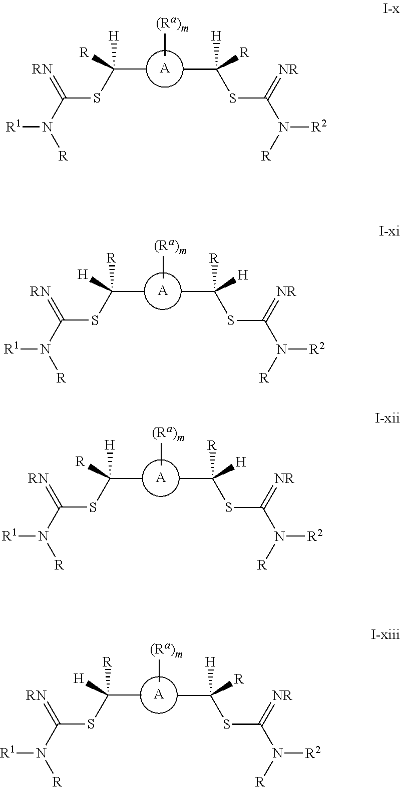

or a pharmaceutically acceptable salt thereof, wherein each of Ring A, R^{a}, m, R¹, R² and R are as defined and described herein.

In some embodiments, a compound of Formula I is of the formula:

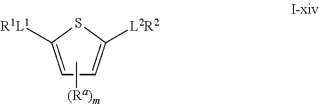

or a pharmaceutically acceptable salt thereof, wherein each of R^{a}, m, R¹, L¹, R², and L² are as defined and described herein. In some embodiments, -L¹R¹ and -L²R² are the same. In some embodiments, -L¹R¹ and -L²R² are different.

In some embodiments, a compound of Formula I is of either of the formulae:

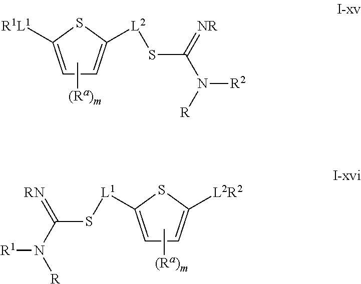

or a pharmaceutically acceptable salt thereof, wherein each of R^{a}, m, R¹, L¹, R², L², and R are as defined and described herein.

In some embodiments, a compound of Formula I is of either of the formulae:

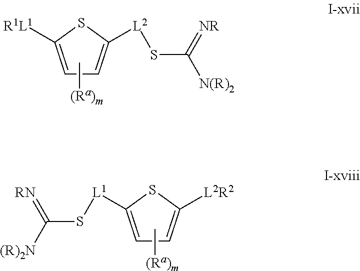

or a pharmaceutically acceptable salt thereof, wherein each of R^{a}, m, R¹, L¹, R², L², and R are as defined and described herein.

In some embodiments, a compound of Formula I is of either of the formulae:

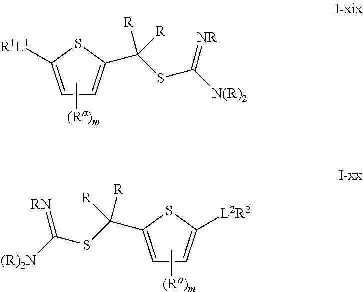

or a pharmaceutically acceptable salt thereof, wherein each of R^{a}, m, R¹, L¹, R², L², and R are as defined and described herein.

In some embodiments, a compound of Formula I is of either of the formulae:

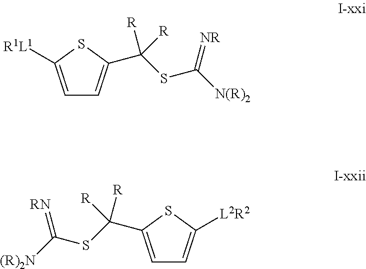

or a pharmaceutically acceptable salt thereof, wherein each of R¹, L¹, R², L², and R are as defined and described herein.

In some embodiments, a compound of Formula I is of the formula:

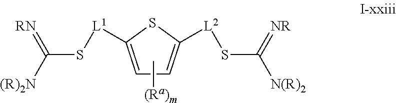

or a pharmaceutically acceptable salt thereof, wherein each of R^{a}, m, L¹, L², and R are as defined and described herein.

In some embodiments, a compound of Formula I is of the formula:

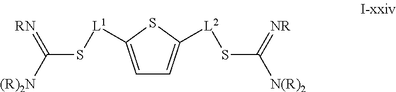

or a pharmaceutically acceptable salt thereof, wherein each of L¹, L², and R are as defined and described herein.

In some embodiments, a compound of Formula I is of the formula:

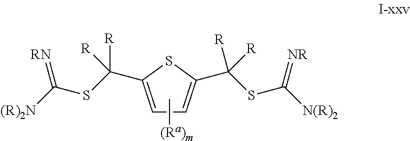

or a pharmaceutically acceptable salt thereof, wherein each of R^{a}, m, and R are as defined and described herein.

In some embodiments, a compound of Formula I is of the formula:

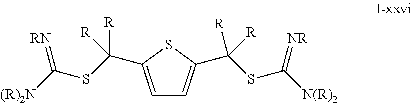

or a pharmaceutically acceptable salt thereof, wherein each R is as defined and described herein.

In some embodiments, a compound of Formula I is of the formula:

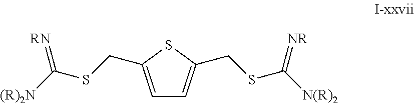

or a pharmaceutically acceptable salt thereof, wherein each R is as defined and described herein.

In some embodiments, a compound of Formula I is of the formula:

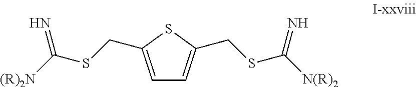

or a pharmaceutically acceptable salt thereof, wherein each R is as defined and described herein.

In some embodiments, a compound of Formula I is of the formula:

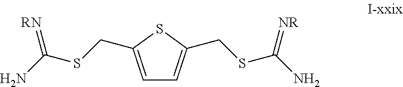

or a pharmaceutically acceptable salt thereof, wherein each R is as defined and described herein.

In some embodiments, a compound of Formula I is of the formula:

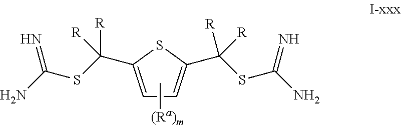

or a pharmaceutically acceptable salt thereof, wherein each of R^{a}, m, and R are as defined and described herein.

In some embodiments, a compound of Formula I is of the formula:

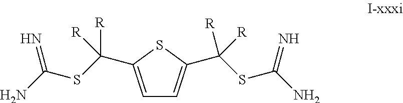

or a pharmaceutically acceptable salt thereof, wherein each R is as defined and described herein.

In some embodiments, a compound of Formula I is of the following structure:

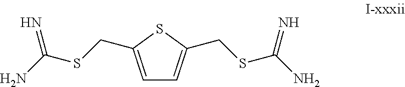 [SMILES: N=C(N)SCc1ccc(CSC(=N)N)s1]

or a pharmaceutically acceptable salt thereof. Exemplary such pharmaceutically acceptable salts are described above and herein. In certain embodiments, the pharmaceutically acceptable salt is a dihydrochloride salt. In certain embodiments, a compound of Formula I is as depicted below:

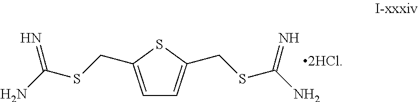 [SMILES: N=C(N)SCc1ccc(CSC(=N)N)s1]

In some embodiments, a compound of Formula I is of any of the following structures:

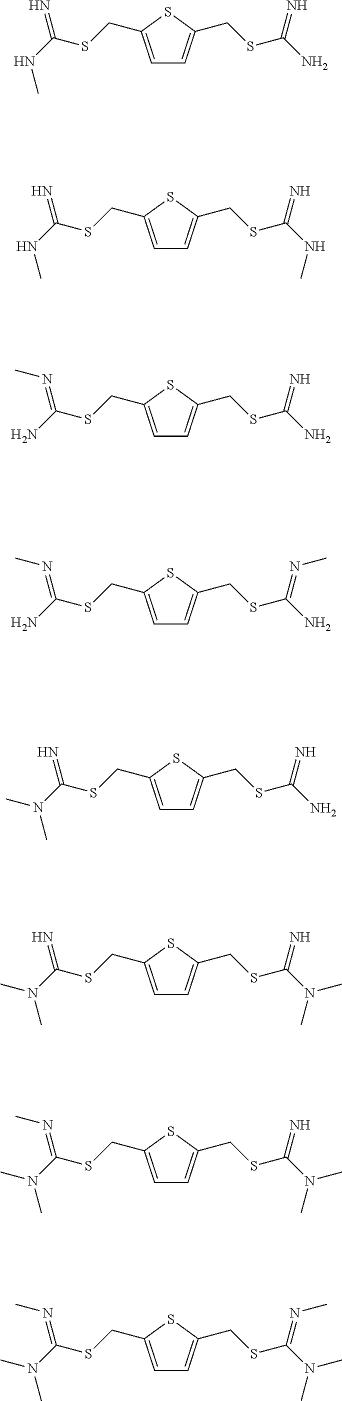

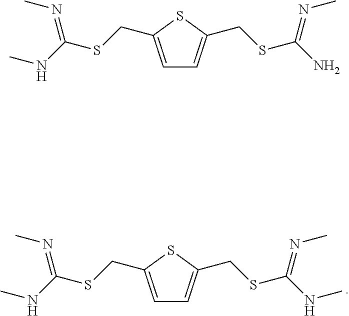

While the above embodiment depicts N-methylated compounds of Formula I, one of skill in the art would appreciate that a variety of N-alkylated compounds are accessible via N-alkylation with a suitable N-alkylating reagent and are contemplated herein. For instance, in some embodiments, N-alkylation comprises N-methylation, M-ethylation, and the like.

In some embodiments, compounds utilized in accordance with the present invention are characterized by and/or are administered under conditions and/or according to a regimen that achieves a reduction in levels of amyloid. In some such embodiments, administration of a compound provided herein to an organism reduces levels of amyloid in one or more particular tissues of interest. In some embodiments, the target tissues are, or include, brain. In some embodiments, amyloid levels are reduced at least 90%, 85%, 80%, 75%, 70%, 65%, 60%, 55%, 50%, 45%, 40%, 35%, 30%, 25%, 20%, 15%, 10%, 5% or more.

In some embodiments, compounds provided herein are characterized by and/or are administered under conditions and/or according to a regimen that achieves one or more of alteration of protein folding pathways, reduction of protein aggregation, alteration of protein degradation pathways, etc. In some embodiments, such alterations stimulate the relevant pathways. In some embodiments, such alterations inhibit the relevant pathways.

In some embodiments, a compound of Formula I shows a maximum tolerated dose (e.g., when tested in a model organism such as a mouse) of at least about 35 to 65 mg/kg IP. In some embodiments, a compound of Formula I shows a maximum tolerated dose of greater than about 40 to about 60 mg/kg IP. In certain embodiments, a compound of Formula I shows a maximum tolerated dose of at least about 50 mg/kg IP.

In some embodiments, a compound of Formula I has a therapeutic index of at least about 5 to about 15. In some embodiments, a compound of Formula I has a therapeutic index of about 8 to about 12. In some embodiments, a compound of Formula I has a therapeutic index of about 10.

In some embodiments, a compound of Formula I exhibits EC₅₀ values of less than about 10 μM.

### General Methods of Preparing Compounds of Formula I

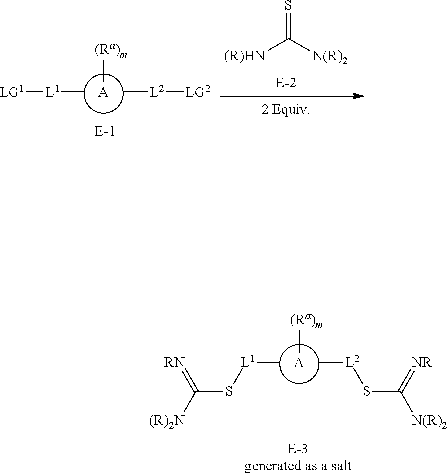

In some embodiments, a compound of Formula I is prepared by reacting a compound of formula E-1, wherein LG¹ and LG² denote suitable leaving groups, with a thiourea of formula E-2 to provide product E-3 as the corresponding salt. That is, in certain embodiments, LG¹ and LG² are halides (e.g., chlorides) and E-3 is obtained as the dihydrohalide (e.g., dihydrochloride) salt. Each of Ring A, R^{a}, m, L¹, L², and R are as defined above and herein.

LG¹ and LG² are each independently a suitable leaving group. Suitable leaving groups are well known in the art, e.g., see, “Advanced Organic Chemistry,” Jerry March, 5^{th} Ed., pp. 351-357, John Wiley and Sons, N.Y. Such leaving groups include, but are not limited to, halogen, alkoxy, sulphonyloxy, optionally substituted alkylsulphonyloxy, optionally substituted alkenylsulfonyloxy, optionally substituted arylsulfonyloxy, and diazonium moieties. Exemplary suitable leaving groups include chloro, iodo, bromo, fluoro, methanesulfonyl (mesyl), tosyl, triflate, nitro-phenylsulfonyl (nosyl), and bromo-phenylsulfonyl (brosyl). In certain embodiments, LG¹ and LG² are each independently halogen. In certain embodiments, LG¹ and LG² are each chloride.

In certain embodiments, the reaction is run in a polar protic solvent such as an alcoholic solvent. Exemplary such solvents include methanol or ethanol.

In certain embodiments, the reaction is run at reflux. In certain embodiments, the reaction is run at ambient temperature.

In some embodiments, the reaction is run for less than about one hour. In some embodiments, the reaction is run for about one, two, three, four, or five hours. In some embodiments, the reaction is run for more than about five hours.

In some embodiments, E-3 is

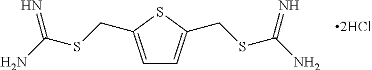 [SMILES: N=C(N)SCc1ccc(CSC(=N)N)s1]

and is synthesized from the reaction of 2,5-bis(chloromethyl)thiophene with two equivalents of thiourea under suitable conditions to provide E-3. In certain embodiments, E-3 is provided in greater than about 50% yield. In certain embodiments, E-3 is provided in greater than about 60% yield. In certain embodiments, E-3 is provided in greater than about 70% yield. In certain embodiments, E-3 is provided in greater than about 80% yield. In certain embodiments, E-3 is provided in greater than about 90% yield. In certain embodiments, E-3 is provided in greater than about 95% yield.

### Pharmaceutical Compositions

Agents of the invention are often administered as pharmaceutical compositions comprising an active therapeutic agent, and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, the carrier or diluent is not saline.

In some embodiments, the present invention provides pharmaceutically acceptable compositions comprising a therapeutically effective amount of one or more of a described compound, formulated together with one or more pharmaceutically acceptable carriers (additives) and/or diluents for use in treating or preventing a disease, disorder, or condition associated with amyloidosis. As described in detail, pharmaceutical compositions of the present invention may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

Pharmaceutically acceptable salts of compounds described herein include conventional nontoxic salts or quaternary ammonium salts of a compound, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. In some embodiments, compounds of the present invention are mixed salts. For instance, in some embodiments, compounds of the present invention comprise two different counterions, such as two different halide ions (e.g., a chloride and a bromide ion), a halide and a sulfonate counterion, a carboxylate and a sulfonate counterion, and the like. One of skill in the art would recognize that a variety of salt combinations exist and, based on the teachings herein, would understand how to make the above exemplary combinations and/or other combinations.

In other cases, described compounds may contain one or more acidic functional groups and, thus, are capable of forming pharmaceutically-acceptable salts with pharmaceutically-acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. See, for example, Berge et al., supra.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations for use in accordance with the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%.

In certain embodiments, a formulation as described herein comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a described compound of the present invention.

Methods of preparing formulations or compositions comprising described compounds include a step of bringing into association a compound of the present invention with the carrier and, optionally, one or more accessory ingredients. In general, formulations may be prepared by uniformly and intimately bringing into association a compound of the present invention with liquid carriers, or finely divided solid carriers, or both, and then, if necessary, shaping the product.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compounds described herein may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming and preservative agents.

Suspensions, in addition to active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Dosage forms for topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Such compositions may also contain adjuvants such as preservatives, wetting agents, emulsifying agents and dispersing agents. Inclusion of one or more antibacterial and/or and antifungal agents, for example, paraben, chlorobutanol, phenol sorbic acid, and the like, may be desirable in certain embodiments. It may alternatively or additionally be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the described compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In certain embodiments, a described compound or pharmaceutical preparation is administered orally. In other embodiments, a described compound or pharmaceutical preparation is administered intravenously. Alternative routs of administration include sublingual, intramuscular, and transdermal administrations.

When compounds described herein are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

Preparations described herein may be given orally, parenterally, topically, or rectally. They are of course given in forms suitable for the relevant administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

Such compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, compounds described herein which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of the invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of described compounds employed in the pharmaceutical composition at levels lower than that required to achieve the desired therapeutic effect and then gradually increasing the dosage until the desired effect is achieved.

In some embodiments, one or more described compounds, or pharmaceutical compositions thereof, is provided to a subject chronically. Chronic treatments include any form of repeated administration for an extended period of time, such as repeated administrations for one or more months, between a month and a year, one or more years, or longer. In many embodiments, chronic treatment involves administering one or more described compounds, or pharmaceutical compositions thereof, repeatedly over the life of the subject. Preferred chronic treatments involve regular administrations, for example one or more times a day, one or more times a week, or one or more times a month. In general, a suitable dose such as a daily dose of one or more described compounds, or pharmaceutical compositions thereof, will be that amount of the one or more described compound that is the lowest dose effective to produce a therapeutic effect. Such an effective dose will generally depend upon the factors described above. Generally doses of the compounds of this invention for a patient, when used for the indicated effects, will range from about 0.0001 to about 100 mg per kg of body weight per day. Preferably, the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be modified as the physiology of the subject changes due to age, disease progression, weight, or other factors.

If desired, the effective daily dose of one or more described compounds may be administered as two, three, four, five, six, or more sub-doses administered separately at appropriate intervals throughout the day, optionally, in unit dosage forms.

While it is possible for a described compound to be administered alone, it is preferable to administer a described compound as a pharmaceutical formulation (composition) as described above.

Described compounds may be formulated for administration in any convenient way for use in human or veterinary medicine, by analogy with other pharmaceuticals.

According to the invention, described compounds for treating neurological conditions or diseases can be formulated or administered using methods that help the compounds cross the blood-brain barrier (BBB). The vertebrate brain (and CNS) has a unique capillary system unlike that in any other organ in the body. The unique capillary system has morphologic characteristics which make up the blood-brain barrier (BBB). The blood-brain barrier acts as a system-wide cellular membrane that separates the brain interstitial space from the blood.

The unique morphologic characteristics of the brain capillaries that make up the BBB are: (a) epithelial-like high resistance tight junctions which literally cement all endothelia of brain capillaries together, and (b) scanty pinocytosis or transendothelial channels, which are abundant in endothelia of peripheral organs. Due to the unique characteristics of the blood-brain barrier, hydrophilic drugs and peptides that readily gain access to other tissues in the body are barred from entry into the brain or their rates of entry and/or accumulation in the brain are very low.

Various strategies have been developed for introducing those drugs into the brain which otherwise would not cross the blood-brain barrier. Widely used strategies involve invasive procedures where the drug is delivered directly into the brain. One such procedure is the implantation of a catheter into the ventricular system to bypass the blood-brain barrier and deliver the drug directly to the brain. These procedures have been used in the treatment of brain diseases which have a predilection for the meninges, e.g., leukemic involvement of the brain (U.S. Pat. No. 4,902,505, incorporated herein in its entirety by reference).

Although invasive procedures for the direct delivery of drugs to the brain ventricles have experienced some success, they are limited in that they may only distribute the drug to superficial areas of the brain tissues, and not to the structures deep within the brain. Further, the invasive procedures are potentially harmful to the patient.

Other approaches to circumventing the blood-brain barrier utilize pharmacologic-based procedures involving drug latentiation or the conversion of hydrophilic drugs into lipid-soluble drugs. The majority of the latentiation approaches involve blocking the hydroxyl, carboxyl and primary amine groups on the drug to make it more lipid-soluble and therefore more easily able to cross the blood-brain barrier.

Another approach to increasing the permeability of the BBB to drugs involves the intra-arterial infusion of hypertonic substances which transiently open the blood-brain barrier to allow passage of hydrophilic drugs. However, hypertonic substances are potentially toxic and may damage the blood-brain barrier.

Antibodies are another method for delivery of compositions of the invention. For example, an antibody that is reactive with a transferrin receptor present on a brain capillary endothelial cell, can be conjugated to a neuropharmaceutical agent to produce an antibody-neuropharmaceutical agent conjugate (U.S. Pat. No. 5,004,697, incorporated herein in its entirety by reference). Such methods are conducted under conditions whereby the antibody binds to the transferrin receptor on the brain capillary endothelial cell and the neuropharmaceutical agent is transferred across the blood brain barrier in a pharmaceutically active form. The uptake or transport of antibodies into the brain can also be greatly increased by cationizing the antibodies to form cationized antibodies having an isoelectric point of between about 8.0 to 11.0 (U.S. Pat. No. 5,527,527, incorporated herein in its entirety by reference).

A ligand-neuropharmaceutical agent fusion protein is another method useful for delivery of compositions to a host (U.S. Pat. No. 5,977,307, incorporated herein in its entirety by reference). The ligand is reactive with a brain capillary endothelial cell receptor. The method is conducted under conditions whereby the ligand binds to the receptor on a brain capillary endothelial cell and the neuropharmaceutical agent is transferred across the blood brain barrier in a pharmaceutically active form. In some embodiments, a ligand-neuropharmaceutical agent fusion protein, which has both ligand binding and neuropharmaceutical characteristics, can be produced as a contiguous protein by using genetic engineering techniques. Gene constructs can be prepared comprising DNA encoding the ligand fused to DNA encoding the protein, polypeptide or peptide to be delivered across the blood brain barrier. The ligand coding sequence and the agent coding sequence are inserted in the expression vectors in a suitable manner for proper expression of the desired fusion protein. The gene fusion is expressed as a contiguous protein molecule containing both a ligand portion and a neuropharmaceutical agent portion.

The permeability of the blood brain barrier can be increased by administering a blood brain barrier agonist, for example bradykinin (U.S. Pat. No. 5,112,596, incorporated herein in its entirety by reference), or polypeptides called receptor mediated permeabilizers (RMP) (U.S. Pat. No. 5,268,164, incorporated herein in its entirety by reference). Exogenous molecules can be administered to the host's bloodstream parenterally by subcutaneous, intravenous or intramuscular injection or by absorption through a bodily tissue, such as the digestive tract, the respiratory system or the skin. The form in which the molecule is administered (e.g., capsule, tablet, solution, emulsion) depends, at least in part, on the route by which it is administered. The administration of the exogenous molecule to the host's bloodstream and the intravenous injection of the agonist of blood-brain barrier permeability can occur simultaneously or sequentially in time. For example, a therapeutic drug can be administered orally in tablet form while the intravenous administration of an agonist of blood-brain barrier permeability is given later (e.g., between 30 minutes later and several hours later). This allows time for the drug to be absorbed in the gastrointestinal tract and taken up by the bloodstream before the agonist is given to increase the permeability of the blood-brain barrier to the drug. On the other hand, an agonist of blood-brain barrier permeability (e.g., bradykinin) can be administered before or at the same time as an intravenous injection of a drug. Thus, the term “co-administration” is used herein to mean that the agonist of blood-brain barrier and the exogenous molecule will be administered at times that will achieve significant concentrations in the blood for producing the simultaneous effects of increasing the permeability of the blood-brain barrier and allowing the maximum passage of the exogenous molecule from the blood to the cells of the central nervous system.

In other embodiments, a described compound can be formulated as a prodrug with a fatty acid carrier (and optionally with another neuroactive drug). The prodrug is stable in the environment of both the stomach and the bloodstream and may be delivered by ingestion. The prodrug passes readily through the blood brain barrier. The prodrug preferably has a brain penetration index of at least two times the brain penetration index of the drug alone. Once in the central nervous system, the prodrug, which preferably is inactive, is hydrolyzed into the fatty acid carrier and a described compound or analog thereof (and optionally another drug). The carrier preferably is a normal component of the central nervous system and is inactive and harmless. The compound and/or drug, once released from the fatty acid carrier, is active. Preferably, the fatty acid carrier is a partially-saturated straight chain molecule having between about 16 and 26 carbon atoms, and more preferably 20 and 24 carbon atoms. Examples of fatty acid carriers are provided in U.S. Pat. Nos. 4,939,174; 4,933,324; 5,994,932; 6,107,499; 6,258,836; and 6,407,137, the disclosures of which are incorporated herein by reference in their entirety.

Administration of agents of the present invention may be for either prophylactic or therapeutic purposes. When provided prophylactically, the agent is provided in advance of disease symptoms. The prophylactic administration of the agent serves to prevent or reduce the rate of onset of symptoms of diseases, disorders, or conditions featuring amyloids. Exemplary such diseases, disorders, or conditions may include, but are not limited to, Alzheimer's Disease, Diabetes mellitus type 2, Parkinson's Disease, Transmissible spongiform encephalopathy (e.g., bovine spongiform encephalopathy), Huntington's Disease, medullary carninoma of the thyroid, cardiac arrythmias, isolated atrial amyloidosis, atherosclerosis, rheumatoid arthritis, aortic medial amyloid, prolactinomas, familial amyloid polyneuropathy, hereditary non-neuropathic systemic amyloidosis, dialysis related amyloidosis, Finnish amyloidosis, lattice corneal dystrophy, cerebral amyloid angiopathy, cerebral amyloid angiopathy (Icelandic type), systemic AL amyloidosis, sporadic inclusion body mytosis, diffuse Lewy Body Disease, multiple system atrophy (MSA), cortico basal degeneration (CBD), progressive supranuclear palsy (PSP), Lewy Body Disease/Lewy Bosy Dementia/Dementia with Lewy Bodies, pantothenate kinase-associated neurodegeneration (PANK1), and amyotrophic lateral sclerosis (ALS).

When provided therapeutically, the agent is provided at (or shortly after) the onset of the appearance of symptoms of actual disease. In some embodiments, the therapeutic administration of the agent serves to reduce the severity and duration of the disease.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (e.g., adjuvants).

For parenteral administration, agents of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl. Compositions for parenteral administration are typically substantially sterile, substantially isotonic and manufactured under GMP conditions of the FDA or similar body.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249, 1527 (1990) and Hanes, Advanced Drug Delivery Reviews 28, 97-119 (1997). The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein. Alternatively, transdermal delivery can be achieved using a skin path or using transferosomes (Paul et al., Eur. J. Immunol. 25, 3521-24 (1995); Cevc et al., Biochem. Biophys. Acta 1368, 201-15 (1998)).

### Combination Therapies

The compositions provided by the present invention can be employed in combination therapies, meaning that the present compositions can be administered concurrently with, prior to, or subsequent to, one or more other desired therapeutic agents or medical procedures. The particular combination of therapies (therapeutic agents or procedures) to employ in a combination regimen will take into account compatibility of the desired therapeutic agents and/or procedures and the desired therapeutic effect to be achieved. It will also be appreciated that the therapies employed may achieve a desired effect for the same disorder (for example, a compound described herein may be administered concurrently with another therapeutic agent used to treat the same disorder), or they may achieve different effects (e.g., control of any adverse effects).

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a provided compound, an additional therapeutic agent, and a pharmaceutically acceptable carrier, adjuvant, or vehicle.

The amount of additional therapeutic agent present in the compositions of this invention will be no more than the amount that would normally be administered in a composition comprising that therapeutic agent as the only active agent. In certain embodiments, the amount of additional therapeutic agent in the present compositions will range from about 50% to 100% of the amount normally present in a composition comprising that agent as the only therapeutically active agent.

For a comprehensive discussion of updated therapies useful for treating neurodegenerative disorders, see, a list of the FDA approved drugs at http://www.fda.gov, and The Merck Manual, Seventeenth Ed. 1999, the entire contents of which are hereby incorporated by reference.

In some embodiments, the compounds of the present invention are combined with other agents useful for treating neurodegenerative disorders wherein such agents include beta-secretase inhibitors/modulators, gamma-secretase inhibitors/modulators, anti-amyloid antibodies, including humanized monoclonal antibodies aggregation inhibitors, metal chelators, antioxidants, and neuroprotectants and inhibitors/modulators of tau phosphorylation (such as GSK3 or CDK inhibitors/modulators) and/or aggregation.

In some embodiments, compounds of the present invention are combined with gamma secretase modulators. In some embodiments, compounds of the present invention are gamma secretase modulators combined with gamma secretase modulators. Exemplary such gamma secretase modulators include, inter alia, certain NSAIDs and their analogs (see WO01/78721 and US 2002/0128319 and Weggen et al., Nature, 414 (2001) 212-16; Morihara et al., J. Neurochem., 83 (2002), 1009-12; and Takahashi et al., J. Biol. Chem., 278 (2003), 18644-70).

In some embodiments, compounds described herein, or pharmaceutically acceptable compositions thereof, can be administered in combination with one or more treatments for Parkinson's Disease such as L-DOPA/carbidopa, entacapone, ropinrole, pramipexole, bromocriptine, pergolide, trihexephendyl, and amantadine; For example, methods of the present invention can be used in combination with medications for treating PD. Such therapeutic agents include levodopa, carbodopa, levodopa (Sinemet and Sinemet CR), Stalevo (carbodopa, levodopa, and entacapone), anticholinergics (trihexyphenidyl, benztropine mesylate, procyclidine, artane, cogentin), bromocriptidine (Parlodel), pergolide (Permax), ropinirol (Requip), pramipexole (Mirapex), cabergoline (Dostinex), apomorphine (Apokyn), rotigotine (Neupro), Ergolide, Mirapex or Requip.

In some embodiments, described compositions and formulations may be administered in combination with one or more treatments for Parkinson's Disease such as ACR-343, rotigotine(Schwarz), rotigotine patch (UCB), apomorphine (Amarin), apomorphine (Archimedes), AZD-3241 (Astra Zeneca), creatine (Avicena), AV-201 (Avigen), lisuride (Axxonis/Biovail), nebicapone (BIAL Group), apomorphine (Mylan), CERE-120 (Ceregene), melevodopa+carbidopa (Cita Neuropharmaceuticals), piclozotan (Daiichi), GM1 Ganglioside (Fidia Farmaceutici), Altropane (Harvard University), Fluoratec (Harvard University), fipamezole (Juvantia Pharma), istradefylline (Kyowa Hakko Kogyo), GPI-1485 (MGI GP), Neu-120 (Neurim Pharmaceuticals), NGN-9076 (NeuroGeneration Inc), NLX-P101 (Neurologix), AFQ-056 (Novartis), arundic acid (Ono/Merck & Co), COMT inhibitor (Orion), ProSavin (Oxford Biomedica), safinamide (Pharmacia & Upjohn), PYM-50028 (Phytopharm), PTX-200 (Phytix), 123I-iometopane (Research Triangle Institute), SYN-115 (Roche Holding), preladenant (Schering Plough), ST-1535 (Sigma-Tau Ind. Farm), ropinirole (SmithKline Beecham), pardoprunox (Solvay), SPN-803 (Supernus Pharmaceuticals), nitisinone (Syngenta), TAK-065 (Takeda), cell therapy (Titan Pharmaceuticals), PD gene therapy (University of Auckland/Weill Medical College), 18F-AV-133 (University of Michigan), mitoquinone/mitoquinol redox mixture (Antipodean Pharmaceuticals), 99m-Tc-tropantiol (University of Pennsylvania), apomorphine (Vectura), BIIB-014 (Vernalis Group), aplindore (Wyeth), and XP-21279 (XenoPort Inc).

Alternatively or additionally, in some embodiments, described compositions and formulations may be administered in combination with one or more treatments for Alzheimer's disease such as Aricept® and Excelon®. In some embodiments, described compositions and formulations may be administered in combination with one or more treatments for Parkinson's Disease such as ABT-126(Abbott Laboratories), pozanicline (Abbott Laboratories), MABT-5102A (AC Immune), Affitope AD-01 (AFFiRiS GmbH), Affitope AD-02 (AFFiRiS GmbH), davunetide (Allon Therapeutics Inc), nilvadipine derivative (Archer Pharmaceuticals), Anapsos (ASAC Pharmaceutical International AIE), ASP-2535 (Astellas Pharma Inc), ASP-2905 (Astellas Pharma Inc), 11C-AZD-2184 (AstraZeneca plc), 11C-AZD-2995 (AstraZeneca plc), 18F-AZD-4694 (AstraZeneca plc), AV-965 (Avera Pharmaceuticals Inc), AVN-101 (Avineuro Pharmaceuticals Inc), immune globulin intravenous (Baxter International Inc), EVP-6124 (Bayer AG), nimodipine (Bayer AG), BMS-708163 (Bristol-Myers Squibb Co), CERE-110 (Ceregene Inc), CLL-502 (CLL Pharma), CAD-106 (Cytos Biotechnology AG), mimopezil ((Debiopharm SA), DCB-AD1 (Development Centre for Biotechnology), EGb-761 ((Dr Willmar Schwabe GmbH & Co), E-2012 (Eisai Co Ltd), ACC-001(Elan Corp plc), bapineuzumab (Elan Corp plc), ELND-006(Elan Pharmaceuticals Inc), atomoxetine (Eli Lilly & Co), LY-2811376 (Eli Lilly & Co), LY-451395 (Eli Lilly & Co), m266 (Eli Lilly & Co), semagacestat (Eli Lilly & Co), solanezumab (Eli Lilly & Co), AZD-103 (Ellipsis Neurotherapeutics Inc), FGLL (ENKAM Pharmaceuticals A/S), EHT-0202 (ExonHit Therapeutics SA), celecoxib (GD Searle & Co), GSK-933776A (GlaxoSmithKline plc), rosiglitazone XR (GlaxoSmithKline plc), SB-742457(GlaxoSmithKline plc), R-1578 (Hoffmann-La Roche AG), HF-0220 (Hunter-Fleming Ltd), oxiracetam (ISF Societa Per Azioni), KD-501 (Kwang Dong Pharmaceutical Co Ltd), NGX-267 (Life Science Research Israel), huperzine A (Mayo Foundation), Dimebon (Medivation Inc), MEM-1414 (Memory Pharmaceuticals Corp), MEM-3454 (Memory Pharmaceuticals Corp), MEM-63908 (Memory Pharmaceuticals Corp), MK-0249 (Merck & Co Inc), MK-0752 (Merck & Co Inc), simvastatin (Merck & Co Inc), V-950 (Merck & Co Inc), memantine (Merz & Co GmbH), neramexane (Merz & Co GmbH), Epadel (Mochida Pharmaceutical Co Ltd), 123I-MNI-330 (Molecular Neuroimaging Llc), gantenerumab (MorphoSys AG), NIC5-15 (Mount Sinai School of Medicine), huperzine A (Neuro-Hitech Inc), OXIGON (New York University), NP-12 (Noscira SA), NP-61 (Noscira SA), rivastigmine (Novartis AG), ECT-AD (NsGene A/S), arundic acid (Ono Pharmaceutical Co Ltd), PF-3084014 (Pfizer Inc), PF-3654746 (Pfizer Inc), RQ-00000009 (Pfizer Inc), PYM-50028 (Phytopharm plc), Gero-46(PN Gerolymatos SA), PBT-2 (Prana Biotechnology Ltd), PRX-03140 (Predix Pharmaceuticals Inc), Exebryl-1(ProteoTech Inc), PF-4360365 (Rinat Neuroscience Corp), HuCAL anti-beta amyloid monoclonal antibodies (Roche AG), EVT-302 (Roche Holding AG), nilvadipine (Roskamp Institute), galantamine (Sanochemia Pharmazeutika AG), SAR-110894 (sanofi-aventis), INM-176 (Scigenic & Scigen Harvest), mimopezil (Shanghai Institute of Materia Medica of the Chinese Academy of Sciences), NEBO-178 (Stegram Pharmaceuticals), SUVN-502 (Suven Life Sciences), TAK-065 (Takeda Pharmaceutical), ispronicline (Targacept Inc), rasagiline (Teva Pharmaceutical Industries), T-817MA (Toyama Chemical), PF-4494700 (TransTech Pharma Inc), CX-717 (University of California), 18F-FDDNP (University of California Los Angeles), GTS-21 (University of Florida), 18F-AV-133 (University of Michigan), 18F-AV-45 (University of Michigan), tetrathiomolybdate (University of Michigan), 123I-IMPY (University of Pennsylvania), 18F-AV-1/ZK (University of Pennsylvania), 11C-6-Me-BTA-1 (University of Pittsburgh), 18F-6-OH-BTA-1 (University of Pittsburgh), MCD-386 (University of Toledo), leuprolide acetate implant (Voyager Pharmaceutical Corp), aleplasinin (Wyeth), begacestat (Wyeth), GSI-136 (Wyeth), NSA-789 (Wyeth), SAM-531 (Wyeth), CTS-21166 (Zapaq), and ZSET-1446 (Zenyaku Kogyo).

Alternatively or additionally, in some embodiments, described compositions and formulations may be administered in combination with one or more treatments for motor neuronal disorders, such as AEOL-10150 (Aeolus Pharmaceuticals Inc), riluzole (Aventis Pharma AG), ALS-08 (Avicena Group Inc), creatine (Avicena Group Inc), arimoclomol (Biorex Research and Development Co), mecobalamin (Eisai Co Ltd), talampanel (Eli Lilly & Co), R-7010 (F Hoffmann-La Roche Ltd), edaravone (Mitsubishi-Tokyo Pharmaceuticals Inc), arundic acid (Ono Pharmaceutical Co Ltd), PYM-50018 (Phytopharm plc), RPI-MN (ReceptoPharm Inc), SB-509 (Sangamo BioSciences Inc), olesoxime (Trophos SA), sodium phenylbutyrate (Ucyclyd Pharma Inc), and R-pramipexole (University of Virginia).

In an alternate embodiment, the methods of this invention that utilize compositions that do not contain an additional therapeutic agent, comprise the additional step of separately administering to said patient an additional therapeutic agent. When these additional therapeutic agents are administered separately they may be administered to the patient prior to, sequentially with or following administration of the compositions of this invention.

### Neurodegenerative Diseases

Imbalances in protein homeostasis are often associated with protein misfolding and/or protein conformational changes that lead to protein aggregation and formation of protein inclusion bodies. Many neurodegenerative diseases, including the polyglutamine (polyQ)-repeat diseases, Alzheimer's disease, Parkinson's disease, prion diseases, frontotemporal lobar degeneration, and ALS, are characterized by the appearance of damaged and aggregated proteins, including huntingtin, polyQ proteins, amyloid A prion (PrP and Sup35) fibrils, and mutant SOD1 (Taylor et al., Science. 2002, 296(5575), 1991-5; Ross, C. A., Neuron. 1997, 19(6), 1147-50; Perutz, M. F., Brain Res Bull. 1999, 50(5-6), 467; and Kopito et al., Nat Cell Bio. 2000, 2(11), E207-9). The fact that such diverse proteins form aggregates in patients with distinct neurological diseases suggests that a common molecular etiology may contribute to the neuropathology in these diseases and that, perhaps, protein misfolding and the subsequent appearance of protein aggregates are early events that play a role in neuronal toxicity in multiple human neurological diseases (Orr, H. T., Genes Dev. 2001, 15(8), 925-32; Ikeda et al., Nat Genet. 1996, 13(2), 196-202; DiFiglia et al., Science. 1997, 277(5334), 1990-3; Davies et al., Cell. 1997, 90(3), 537-48; Koo et al., Proc Nall Acad Sci USA. 1999, 96(18), 9989-90).

Methods of Using Compounds of Formula I in Accordance with the Present Invention

The present invention encompasses the recognition that compounds of Formula I can be effective in treating patients with neurodegenerative diseases, disorders, or conditions featuring amyloids. Exemplary such diseases, disorders, or conditions may include, but are not limited to, Alzheimer's Disease, Diabetes mellitus type 2, Parkinson's Disease, Transmissible spongiform encephalopathy (e.g., bovine spongiform encephalopathy), Huntington's Disease, medullary carninoma of the thyroid, cardiac arrythmias, isolated atrial amyloidosis, atherosclerosis, rheumatoid arthritis, aortic medial amyloid, prolactinomas, familial amyloid polyneuropathy, hereditary non-neuropathic systemic amyloidosis, dialysis related amyloidosis, Finnish amyloidosis, lattice corneal dystrophy, cerebral amyloid angiopathy, cerebral amyloid angiopathy (Icelandic type), systemic AL amyloidosis, sporadic inclusion body mytosis, diffuse Lewy Body Disease, multiple system atrophy (MSA), cortico basal degeneration (CBD), progressive supranuclear palsy (PSP), Lewy Body Disease/Lewy Bosy Dementia/Dementia with Lewy Bodies, pantothenate kinase-associated neurodegeneration (PANK1), and amyotrophic lateral sclerosis (ALS).

In certain embodiments, the neurodegenerative disease is Alzheimer's Disease.

In certain embodiments, the neurodegenerative disease is Parkinson's Disease.

The invention provides methods for treating a subject suffering from or susceptible to a neurodegenerative disease, disorder, or condition featuring amyloids including the step of administering to the subject a therapeutically effective amount of a compound of Formula I or a pharmaceutical composition thereof. In certain embodiments, the subject is a mouse. In certain embodiments, the subject is an adult human.

In some embodiments, the present invention provides a method for treating diseases where A-beta amyloidosis is an underlying aspect or a co-existing and exacerbating factor, wherein said method comprises administering to said patient a compound of Formula I, or a pharmaceutically acceptable composition thereof.

In some embodiments, the present invention provides pharmaceutical composition comprising: a retromer-stabilizing agent and a pharmaceutically acceptable carrier,

wherein the retromer-stabilizing agent specifically binds to a conformational-specific target corresponding to an interface formed by at least two components of the retromer, wherein said at least two components includes VPS35; and

wherein the retromer-stabilizing agent is a compound of Formula I:

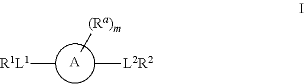

or a pharmaceutically acceptable salt thereof, wherein each of Ring A, m, R¹, L¹, R², and L² are as defined and described herein.

In some embodiments, the present invention provides a complex comprising retromer and a retromer-stabilizing agent,

wherein the retromer-stabilizing agent is a compound of Formula I:

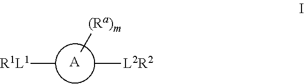

or a pharmaceutically acceptable salt thereof, wherein each of Ring A, m, R¹, L¹, R², and L² are as defined and described herein. In some embodiments, the retromer is stabilized by the retromer-stabilizing agent such that the thermal stability of the complex is increased by at least about 5° C., as compared to that of retromer without the retromer-stabilizing agent. In some embodiments, the thermal stability of the complex is increased by at least about 6° C., about 7° C., about 8° C., about 9° C., or about 10° C., as compared to that of retromer without the retromer-stabilizing agent. In some embodiments, the retromer comprises at least one retromer component that contains at least one mutation. In certain embodiments, said at least one retromer component is VPS35, VPS29, VPS26 or combination thereof.

In some embodiments, the present invention provides a method for stabilizing retromer in a cell, the method comprising a step of:

contacting a cell expressing retromer components with a retromer-stabilizing agent in an amount effective to increase the stability of retromer complex,

wherein the retromer-stabilizing agent is a compound of Formula I:

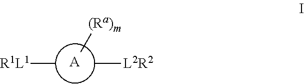

or a pharmaceutically acceptable salt thereof, wherein each of Ring A, m, R¹, L¹, R², and L² are as defined and described herein. In some embodiments, the cell expresses a VPS35 mutant polypeptide containing one or more mutations that destabilize retromer. In certain embodiments, said one or more mutations occur at amino acid residue(s) 534, 541, 579, 582, 586, 589, 629, 630, 633, 637, 672, 675, 725, 729, 769, 772, 776 or any combinations thereof.

In some embodiments, the present invention provides a method for treating amyloidosis, the method comprising a step of:

administering to a subject having or susceptible to developing amyloidosis a pharmaceutical composition comprising a retromer-stabilizing agent and a pharmaceutically acceptable carrier, wherein the retromer-stabilizing agent is a compound of Formula I:

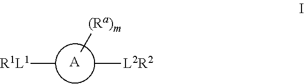

or a pharmaceutically acceptable salt thereof, wherein each of Ring A, m, R¹, L¹, R², and L² are as defined and described herein.

In some embodiments, the present invention provides a method for reducing amyloid accumulation in a subject, the method comprising a step of:

administering to a subject having amyloid accumulation a pharmaceutical composition comprising a retromer-stabilizing agent and a pharmaceutically acceptable carrier, in an amount effective to reduce amyloid accumulation in the subject, wherein the retromer-stabilizing agent is a compound of Formula I:

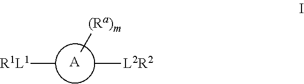

or a pharmaceutically acceptable salt thereof, wherein each of Ring A, m, R¹, L¹, R², and L² are as defined and described herein. In some embodiments, the subject expresses a VPS35 mutant polypeptide containing one or more mutations. In certain embodiments, said one or more mutations occur at amino acid residue(s) 534, 541, 579, 582, 586, 589, 629, 630, 633, 637, 672, 675, 725, 729, 769, 772, 776 or any combinations thereof.

In some embodiments, the present invention provides a method for promoting retromer-mediated endosome-TGN trafficking of a protein in a cell, the method comprising:

contacting a cell expressing retromer components with a retromer-stabilizing agent in an amount effective to promote retromer-mediated trafficking of a protein from the endosome to the TGN of the cell, wherein the retromer-stabilizing agent is a compound of Formula I:

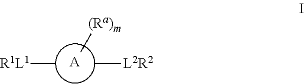

or a pharmaceutically acceptable salt thereof, wherein each of Ring A, m, R¹, L¹, R², and L² are as defined and described herein. In some embodiments, the retromer-stabilizing agent binds to a binding site of a VPS35/VPS29 binary complex, wherein the binding site is at an interface of the VPS35/VPS29 binary complex in a crystal structure; and, wherein the interface involves one or more of the following amino acid residues corresponding to wild type VPS29: D8, H10, R14, N39, D62, F63, H86, I91, P92, W93, G94, L101, R104, H115, Y139, A141 and L142. In certain embodiments, the retromer-stabilizing agent binds to a binding site (e.g., target) of a VPS35/VPS29 binary complex, wherein the binding site (e.g., target) is located at an interface of the VPS35/VPS29 binary complex in a crystal structure; and, wherein the interface involves one or more of the following amino acid residues corresponding to wild type VPS35: F534, F541, L579, R582, Q586, L589, T629, L630, G633, R637, T672, H675, N725, Y729, H769, N772 and H776. In some embodiments, such an interface comprises a target site for the agent as set forth as I-xxxiv (see below).

In some embodiments, retromer stabilizing agents of the present invention preferentially bind to retromer containing at least one mutation. In some embodiments, retromer stabilizing agents of the present invention preferentially bind to retromer containing at least one mutation that alters the three-dimensional conformation of the complex. In some embodiments, retromer stabilizing agents of the present invention preferentially bind to retromer containing at least one mutation that alters the three-dimensional conformation (e.g., misfolding) of the complex such that the mutation or mutations cause structural destabilization of the complex.

In some embodiments, the present invention provides any one of the above-described methods and the retromer-stabilizing agent is:

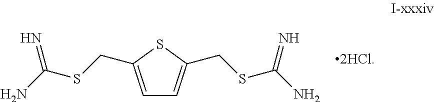 [SMILES: N=C(N)SCc1ccc(CSC(=N)N)s1]

In some embodiments, the cell expressing retromer components is a neuronal cell. In some embodiments, the neuronal cell is a hippocampal neuron. In some embodiments, the hippocampal neuron is localized to the entorhinal cortex. In some embodiments, the protein is a type 1 transmembrane receptor. In certain embodiments, the type 1 transmembrane receptor contains a VPS10-domain. In certain embodiments, examples of type 1 transmembrane receptors include, but are not limited to: sorLA, sortilin, SorCS1, SorCS2, SorCS3, APP, BASE1, presenilin and Wnt signaling protein.

### Alzheimer's Disease (AD)

As described above, the present invention provides a method for treating or lessening the severity of a disorder associated with amyloidosis. In certain embodiments, the amyloidosis is A-beta amyloidosis. Such disorders also include inclusion body myositis (deposition of A-beta in peripheral muscle, resulting in peripheral neuropathy), cerebral amyloid angiopathy (amyloid in the blood vessels in the brain), and mild cognitive impairment and pre-symptomatic, prodromal or predementia AD.

Alzheimer's Disease (AD) is believed to result from the deposition of quantities of a peptide, amyloid-beta (“A-beta”), within the brain. This peptide is produced by enzymatic cleavage of amyloid protein precursor (“APP”) protein. The C-terminus of A-beta is generated by an enzyme termed gamma-secretase. Cleavage occurs at more than one site on APP producing different length A-beta peptides, some of which are more prone to deposition, such as A-beta 42. It is believed that aberrant production A-beta 42 in the brain leads to AD.

A-beta, a 37-43 amino acid peptide derived by proteolytic cleavage of the amyloid precursor protein (APP), is the major component of amyloid plaques. APP is expressed and constitutively catabolized in most cells. APP has a short half-life and is metabolized rapidly down two pathways. In one pathway, cleavage by an enzyme known as alpha-secretase occurs while APP is still in the trans-Golgi secretory compartment. This cleavage by alpha-secretase occurs within the A-beta portion of APP, thus precluding the formation of A-beta.

In contrast to this non-amyloidogenic pathway involving alpha-secretase described above, proteolytic processing of APP by beta-secretase exposes the N-terminus of A-beta, which after gamma-secretase cleavage at the variable C-terminus, liberates A-beta. Peptides of 40 or 42 amino acids in length (A-beta 1-40 and A-beta 1-42, respectively) predominate among the C-termini generated by gamma-secretase, however, a recent report suggests 1-38 is a dominant species in cerebrospinal fluid. A-beta 1-42 is more prone to aggregation than A-beta 1-40, the major component of amyloid plaque, and its production is closely associated with the development of Alzheimer's disease. The bond cleaved by gamma-secretase appears to be situated within the transmembrane domain of APP. In the amyloidogenic pathway, APP is cleaved by beta-secretase to liberate sAPP-beta and CTF-beta, which CTF-beta is then cleaved by gamma-secretase to liberate the harmful A-beta peptide.

While abundant evidence suggests that extracellular accumulation and deposition of A-beta is a central event in the etiology of AD, recent studies have also proposed that increased intracellular accumulation of A-beta or amyloid containing C-terminal fragments may play a role in the pathophysiology of AD. For example, over-expression of APP harboring mutations which cause familial Alzheimer's disease (AD) results in the increased intracellular accumulation of CTF-beta in neuronal cultures and A-beta 42 in HEK 293 cells.

A-beta 42 is the 42 amino acid long form of A-beta that is believed to be more potent in forming amyloid plaques than the shorter forms of A-beta. Moreover, evidence suggests that intra- and extracellular A-beta are formed in distinct cellular pools in hippocampal neurons and that a common feature associated with two types of familial AD mutations in APP (“Swedish” and “London”) is an increased intracellular accumulation of A-beta 42.

Without wishing to be bound by any particular theory, it is believed that of importance in this A-beta-producing pathway is the position of the gamma-secretase cleavage. If the gamma-secretase proteolytic cut is at residue or before 711-712, shorter A-beta. (A-beta 40 or shorter) is the result; if it is a proteolytic cut after residue 713, long A-beta (A-beta 42) is the result. Thus, the .gamma. secretase process is central to the production of A-beta peptide of 40 or 42 amino acids in length (A-beta 40 and A-beta 42, respectively). For a review that discusses APP and its processing, see Selkoe, 1998, Trends Cell. Biol. 8:447-453; Selkoe, 1994, Ann. Rev. Cell Biol. 10:373-403. See also, Esch et al., 1994, Science 248:1122.

Cleavage of APP can be detected in a number of convenient manners, including the detection of polypeptide or peptide fragments produced by proteolysis. Such fragments can be detected by any convenient means, such as by antibody binding. Another convenient method for detecting proteolytic cleavage is through the use of a chromogenic .beta. secretase substrate whereby cleavage of the substrate releases a chromogen, e.g., a colored or fluorescent, product. More detailed analyses can be performed including mass spectroscopy.

In some embodiments, one or more compounds of the present invention are administered to a patient suffering from mild cognitive impairment or age-related cognitive decline or pre-symptomatic AD or prodromal or predementia AD (Dubois et al The Lancet Neurology 10 (2010) 70223-4). In some embodiments, a favourable outcome of such treatment is prevention or delay of the onset of AD. Age related cognitive decline and mild cognitive impairment (MCI) are conditions in which a memory deficit is present, but other diagnostic criteria for dementia are absent (Santacruz and Swagerty, American Family Physician, 63 (2001), 703-13). As used herein, “age-related cognitive decline” implies a decline of at least six months' duration in at least one of: memory and learning; attention and concentration; thinking; language; and visuospatial functioning and a score of more than one standard deviation below the norm on standardized neuropsychologic testing such as the MMSE.

“High A-beta42” is a measurable condition that precedes symptomatic disease, especially in familial patients, based on plasma, CSF measurements, and/or genetic screening or brain imaging. This concept is analogous to the relationship between elevated cholesterol and heart disease. Thus, another aspect of the present invention provides a method for preventing a disorder associated with elevated amyloid-beta (1-42) peptide, wherein said method comprises administering to said patient a provided compound or a pharmaceutically acceptable composition thereof.

In certain embodiments, the present invention provides a method for treating or lessening the severity of Alzheimer's disease in a patient, wherein said method comprises administering to said patient a compound of Formula I, or a pharmaceutically acceptable composition thereof.

### Parkinson's Disease

Parkinson's disease (PD) is a neurological disorder characterized by bradykinesia, rigidity, tremor, and postural instability. The pathologic hallmark of PD is loss of neurons in the substantia nigra pars compacta (SNpc) and the appearance of Lewy bodies in remaining neurons. It appears that more than about 50% of the cells in the SNpc need to be lost before motor symptoms appear. Associated symptoms often include small handwriting (micrographia), seborrhea, orthostatic hypotension, urinary difficulties, constipation and other gastrointestinal dysfunction, sleep disorders, depression and other neuropsychiatric phenomena, dementia, and smelling disturbances (occurs early). Patients with Parkinsonism have greater mortality, about two times compared to general population without PD. This is attributed to greater frailty or reduced mobility.

Diagnosis of PD is mainly clinical and is based on the clinical findings listed above. Parkinsonism, refers to any combination of two of bradykinesia, rigidity, and/or tremor. PD is the most common cause of parkinsonism. Other causes of parkinsonism are side effects of drugs, mainly the major tranquilizers, such as Haldol, strokes involving the basal ganglia, and other neurodegenerative disorders, such as Diffuse Lewy Body Disease (DLBD), progressive supranuclear palsy (PSP), frontotemporal dementia (FTD), MSA, and Huntington's disease. The pathological hallmark of PD is the Lewy body, an intracytoplasmatic inclusion body typically seen in affected neurons of the substantia nigra and to a variable extent, in the cortex. Recently, α-synuclein has been identified as the main component of Lewy bodies in sporadic Parkinsonism.

Although parkinsonism can be clearly traced to viruses, stroke, or toxins in a few individuals, for the most part, the cause of Parkinson's disease in any particular case is unknown. Environmental influences which may contribute to PD may include drinking well water, farming and industrial exposure to heavy metals (e.g., iron, zinc, copper, mercury, magnesium and manganese), alkylated phosphates, and orthonal chlorines. Paraquat (a herbicide) has also been associated with increased prevalence of Parkinsonism including PD. Cigarette smoking is associated with a decreased incidence of PD. The current consensus is that PD may either be caused by an uncommon toxin combined with high genetic susceptibility or a common toxin combined with relatively low genetic susceptibility.

A small percentage of subjects that are at risk of developing PD can be identified for example by genetic analysis. There is good evidence for certain genetic factors being associated with PD. Large pedigrees of autosomal dominantly inherited PDs have been reported. For example, a mutation in α-synuclein is responsible for one pedigree and triplication of the SNCA gene (the gene coding for α-synuclein) is associated with PD in others.

Methods of the invention can be used in combination with one or more other medications, including medications that are currently used to treat synucleinopathies or symptoms arising as side-effects of the disease or of the aforementioned medications.

For example, methods of the invention can be used in combination with medications for treating PD. Levodopa mainly in the form of combination products containing carbodopa and levodopa (Sinemet and Sinemet CR) is the mainstay of treatment and is the most effective agent for the treatment of PD. Levodopa is a dopamine precursor, a substance that is converted into dopamine by an enzyme in the brain. Carbodopa is a peripheral decarboxylase inhibitor which prevents side effects and lower the overall dosage requirement. The starting dose of Sinemet is a 25/100 or 50/200 tablet prior to each meal. Dyskinesias may result from overdose and also are commonly seen after prolonged (e.g., years) use. Direct acting dopamine agonists may have less of this side effect. About 15% of patients do not respond to levodopa. Stalevo (carbodopa, levodopa, and entacapone) is a new combination formulation for patients who experience signs and symptoms of “wearing-off” The formulation combines carbodopa and levodopa (the most widely used agents to treat PD) with entacapone, a catechol-O-methyltransferase inhibitor. While carbodopa reduces the side effects of levodopa, entacapone extends the time levodopa is active in the brain, up to about 10% longer.

Amantidine (SYMMETREL®) is a mild agent thought to work by multiple mechanisms including blocking the re-uptake of dopamine into presynaptic neurons. It also activates the release of dopamine from storage sites and has a glutamate receptor blocking activity. It is used as early monotherapy, and the dosing is 200 to 300 mg daily. Amantadine may be particularly helpful in patients with predominant tremor. Side effects include ankle swelling and red blotches. It may also be useful in later stage disease to decrease the intensity of drug-induced dyskinesia.

Anticholinergics (trihexyphenidyl, benztropine mesylate, procyclidine, artane, cogentin) do not act directly on the dopaminergic system. Direct-acting dopamine agonists include bromocriptidine (Parlodel), pergolide (Permax), ropinirol (Requip), and pramipexole (Mirapex). These agents cost substantially more than levodopa (Sinemet), and additional benefits are controversial. Depending on which dopamine receptor is being stimulated, D1 and D2 agonist can exert anti-Parkinson effects by stimulating the D1 and D2 receptors, such as Ergolide. Mirapex and Requip are the newer agents. Both are somewhat selected for dopamine receptors with highest affinity for the D2 receptor and also activity at the D3 receptor. Direct dopamine agonists, in general, are more likely to produce adverse neuropsychiatric side effects such as confusion than levodopa. Unlike levodopa, direct dopamine agonists do not undergo conversion to dopamine and thus do not produce potentially toxic free radical as they are metabolized. It is also possible that the early use of direct dopamine agonist decreases the propensity to develop the late complications associated with direct stimulation of the dopamine receptor by dopamine itself, such as the “on-off” effect and dyskinesia.

Monoaminoxidase-B inhibitors (MAO) such as selegiline (Diprenyl, or Eldepryl), taken in a low dose, may reduce the progression of Parkinsonism. These compounds can be used as an adjunctive medication. A study has documented that selegiline delays the need for levodopa by roughly three months, although interpretation of this data is confounded by the mild symptomatic benefit of the drug. Nonetheless, theoretical and in vitro support for a neuroprotective effect for some members of the selectiv MAOB class of inhibitors remains (e.g., rasagiline).

Catechol-O-methyltransferase inhibitors (COMT) can also be used in combination treatments of the invention. Catechol-O-methyltransferase is an enzyme that degrades levodopa, and inhibitors can be used to reduce the rate of degradation. Entacapone is a peripherally acting COMT inhibitor, which can be used in certain methods and compositions of the invention. Tasmar or Tolcapone, approved by the FDA in 1997, can also be used in certain methods and compositions of the invention. Psychiatric adverse effects that are induced or exacerbated by PD medication include psychosis, confusion, agitation, hallucinations, and delusions. These can be treated by decreasing dopamine medication, reducing or discontinuing anticholinergics, amantadine or selegiline or by using low doses of atypical antipsychotics such as clozapine or quetiapine.

### EXEMPLIFICATION

Previously, it was reported that downregulation of the retromer protein complex in a subset of AD patients but not in controls, and showed that reduction of retromer levels raised Abeta levels and induced AD-like symptoms in rodents. We sought to test whether pharmacological stabilization of retromer, which would be expected to raise its steady-state level in the neuron, could be of value in treating AD. We now have identified a compound that does exactly that: it stabilizes retromer in the active complex form, and does nothing to the individual protein components. The compound was initially made for the NCI cancer screening program and had no effect in cancer models, but was shown to be safe in mice at up to 30 mg/kg dose. The invention contemplates that this compound and analogs can be used for stabilizing retromer, thereby raising levels of retromer complex in cells in culture. It is further contemplated that these compositions can be administered to AD mice. The test compound (see below) is easy to synthesize.

Alzheimer Disease begins in the hippocampal formation before sweeping over the neocortex, ravaging the mind and causing dementia in its wake. The hippocampal formation itself, however, is a circuit made up of separate but interconnected subregions—the entorhinal cortex, the dentate gyms, the CA3 and CA1 subfields, and the subiculum. Each hippocampal subregion expresses a unique molecular profile, accounting for why each subregion is differentially vulnerable to mechanisms of disease. During the past few years, variants of functional magnetic resonance imaging (fMRI) have been used to investigate the hippocampus as a circuit—e.g., simultaneously investigating multiple subregions—establishing a spatiotemporal profile of AD-related dysfunction. Agreeing with some, although not all, postmortem indicators of disease, the spatial pattern of dysfunction suggests that, early on, AD targets the entorhinal cortex with relative sparing of the dentate gyms. In contrast to the spatial pattern, the temporal pattern of dysfunction uncovered by the imaging studies was unexpected and could not have been inferred from postmortem indicators alone. Specifically, entorhinal dysfunction detected in early AD was age invariant.

This spatiotemporal profile was used to construct a model predicting how a pathogenic molecule related to AD should behave. Guided by the model, the entorhinal cortex and the dentate gyms from postmortem brain specimens with and without AD were harvested, purposefully covering a broad age span, and microarray analysis was performed on each tissue sample. The final analysis revealed that, among a handful of hits, the expression level of vacuolar protein sorting 35 (VPS35), a component of the retromer protein complex, best conformed to the full spatiotemporal model of late-onset AD.

First described in yeast, the multimeric retromer complex comprises the proteins VPS35, VPS26, VPS29, VPS5, and VPS17. This complex acts as a “coat” that binds and transports the transmembrane receptor VPS10 from the endosome back to the trans-Golgi network 10. Except for VPS17, mammalian homologues of every component of the retromer complex have been identified and are expressed in the brain.

Previous studies have shown that a primary reduction in any retromer element will lead to secondary degradation of other elements of the complex, causing general retromer dysfunction.

Indeed, it has been shown that both VPS35 and VPS26 protein levels were reduced in AD compared with age-matched normal controls. To test whether this finding was potentially relevant to pathogenesis, small interfering RNA was used to systematically decrease retromer elements in cell culture, showing that this reduction led to increased concentrations of Aβ, while overexpressing retromer elements decreased A13 levels. More recently, a genetic study was carried out by Rogaeva et al. Investigating multiple cohorts with late-onset AD, they genotyped VPS35, VPS26, and the family of VPS10-containing molecules. Remarkably, genetic variants in the VPS10-protein sorLA were associated with late-onset AD and not with age-matched normal controls. The researchers also provide direct evidence that VPS35 binds sorLA and that knocking down the VPS35 binding partner VPS26 in cell culture increases Aβ production.

We set out to screen and identify compounds that stabilized the active retromer complex, thereby preventing VPS35 degradation and increasing the steady-state level of the complex, which may reduce toxic Abeta. To cast our net wider, we set out to perform virtual screening by docking first, and we were able to do that because crystal structures of several retromer components and, more importantly, the VPS35-VPS29 binary complex, had been published. However, in order to carry out real stability assays on any in silico “hits”, we needed to obtain purified retromer complex in large quantities, and this proved extremely challenging. After two years of trial and error in overcoming technical obstacles, we finally succeeded in expressing and purifying each of the individual retromer components separately, and then reconstituting the complete complex in vitro.

We were ready for conducting docking To identify candidate docking sites, we used a fragment-based surface mapping technique, first experimentally and then computationally. The computational method was used, which bombards the protein surface with drug fragments in the computer and identifies the stickiest portions of the protein surface as likely ligand binding sites. As a result, seven sites were found.

The library of potential drugs he then docked contains around 50,000 compounds. They were chosen largely on the basis of assumed or actual cell penetration, the absence of cytotoxicity, likely solubility, and no known promiscuous binders. Each was docked to every putative ligand binding site, which took months of computation, and the computed “energy” of interaction (a highly simplified function) was ranked (G-score).

We then obtained tested, in a real fluorescence stability assay using our reconstituted retromer, a total of about 50 compounds from our list of the 200 “top hits” in the docking run. It should be noted that there are a total of 7 sites that were identified from the computational surface mapping approach, and we considered anything with a G-score below −7.00 to be in the “top hits”. Four of the sites contained almost all the top hits, so compounds were actually assayed only if they were in the top 200 by G-score and were predicted to bind well to one of these four sites.

Site 2-87 top hits (7 compounds tested. All positive hits in actual experimental assays, including compound 55712 (see below), have come from the list of top docking hits in this site. The site sits at the interface between VPS35 and VPS29 in the crystal structure of the binary complex.)

Site 3-36 top hits (5 compounds tested)
Site 5-46 top hits (3 compounds tested)
Site 7-16 top hits (3 compounds tested)

Using this method, we can identify additional candidate retromer stabilizing compounds.

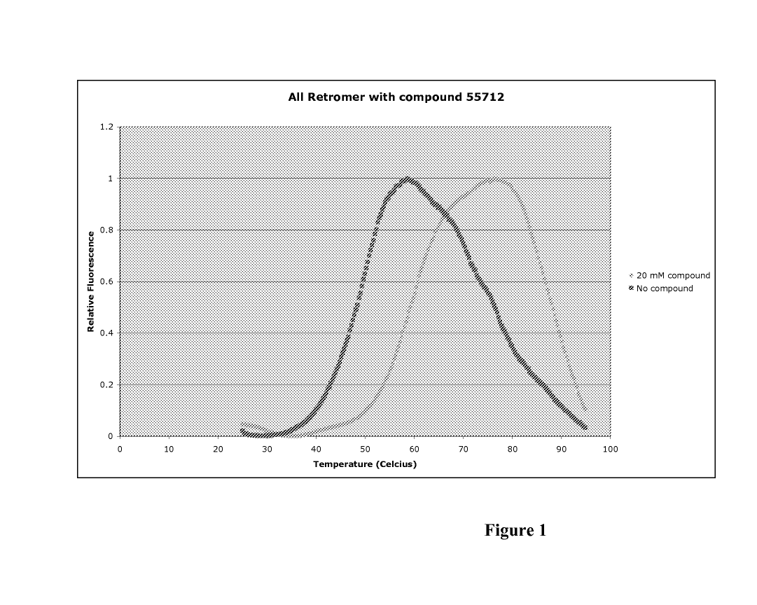

However, as it is, compound 55712, one of the 7 we tested from the list of site 2 predicted binders, stabilized the intact retromer complex by 10 degrees Celsius, a number comparable to that observed for the best pharmacological chaperones currently in clinical trials for other diseases. FIG. 1 shows the thermal stability assay for this compound.

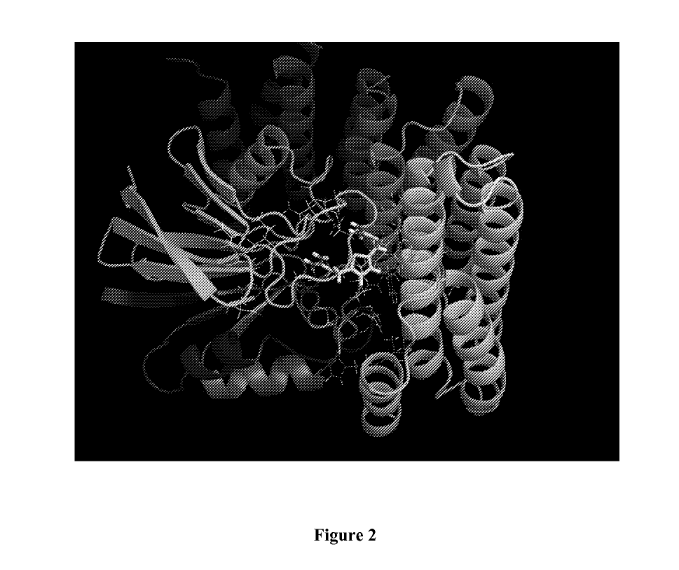

In addition, FIG. 2 shows the structure of the compound docked into site 2 in the VPS35-VPS29 complex crystal structure. VPS 35 is the all-helical subunit.

The compound used for this example was made by the National Cancer Institute as part of their diversity chemical library creation to find new anticancer drugs. It was tested in three different animal models for cancer and showed no antitumor activity, but it was not toxic to mice at concentrations up to 30 mg/kg.

Some details on the compound, which we will call BF301 (Brandeis-Fidelity 301), are given here. As described in more detail, we have devised a one-step synthesis in 95% yield using commercially available starting materials and 1 hr reflux in ethanol at room temperature.

What is claimed is:

1. A pharmaceutical composition comprising: a retromer-stabilizing agent and a pharmaceutically acceptable carrier,
wherein the retromer-stabilizing agent specifically binds to a conformational-specific target corresponding to an interface formed by at least two components of the retromer, wherein said at least two components includes VPS35; and
wherein the retromer-stabilizing agent is a compound of Formula I: 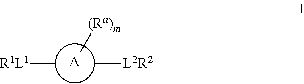
or a pharmaceutically acceptable salt thereof, wherein:
Ring A is an optionally substituted bivalent 3-8 membered saturated, partially unsaturated, or aryl monocyclic ring having 0-4 heteroatoms independently selected from nitrogen, oxygen, or sulfur, or an optionally substituted bivalent 8-10 membered saturated, partially unsaturated, or aryl bicyclic ring having 0-4 heteroatoms independently selected from nitrogen, oxygen, or sulfur;
m is 0-5;
each R^{a} is independently —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂;
each R is independently deuterium, hydrogen, halogen, an optionally substituted C₁₋₆ aliphatic group, or an optionally substituted 3-8 membered saturated, partially unsaturated, or aryl ring having 0-4 heteroatoms independently selected from nitrogen, oxygen, or sulfur, or wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur, or wherein:
two R on the same carbon are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur; and
L¹ and L² are each independently a valence bond or a bivalent optionally substituted C₁₋₁₀ alkylene chain wherein one, two, or three methylene units are optionally and independently replaced by —O—, —N(R)—, —S—, —C(O)—, —C(═NR)— —OC(O)—, —C(O)O—, —OC(O)O—, —S(O)—, —S(O)₂—, —OSO₂O—, —N(R)C(O)—, —C(O)N(R)—, —N(R)C(O)O—, —OC(O)NR—, —N(R)C(O)NR—, and wherein L¹ and L² are each independently optionally substituted with 1-6 R groups; and
R¹ and R² are each independently selected from —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂, —SC(═NR)N(R)₂, or an optionally substituted C₁₋₂₀ aliphatic group.

2. A complex comprising retromer and a retromer-stabilizing agent,
wherein the retromer-stabilizing agent is a compound of Formula I: 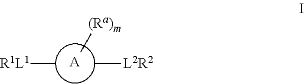
or a pharmaceutically acceptable salt thereof, wherein:
Ring A is an optionally substituted bivalent 3-8 membered saturated, partially unsaturated, or aryl monocyclic ring having 0-4 heteroatoms independently selected from nitrogen, oxygen, or sulfur, or an optionally substituted bivalent 8-10 membered saturated, partially unsaturated, or aryl bicyclic ring having 0-4 heteroatoms independently selected from nitrogen, oxygen, or sulfur;
m is 0-5;
each R^{a} is independently —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂;
each R is independently deuterium, hydrogen, halogen, an optionally substituted C₁₋₆ aliphatic group, or an optionally substituted 3-8 membered saturated, partially unsaturated, or aryl ring having 0-4 heteroatoms independently selected from nitrogen, oxygen, or sulfur, or wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur, or wherein:
two R on the same carbon are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur; and
L¹ and L² are each independently a valence bond or a bivalent optionally substituted C₁₋₁₀ alkylene chain wherein one, two, or three methylene units are optionally and independently replaced by —O—, —N(R)—, —S—, —C(O)—, —C(═NR)— —OC(O)—, —C(O)O—, —OC(O)O—, —S(O)—, —S(O)₂—, —OSO₂O—, —N(R)C(O)—, —C(O)N(R)—, —N(R)C(O)O—, —OC(O)NR—, —N(R)C(O)NR—, and wherein L¹ and L² are each independently optionally substituted with 1-6 R groups; and
R¹ and R² are each independently selected from —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂, —SC(═NR)N(R)₂, or an optionally substituted C₁₋₂₀ aliphatic group.

3. The complex of claim 2, wherein the retromer is stabilized by the retromer-stabilizing agent in that the thermal stability of the complex is increased by at least about 5° C., as compared to that of retromer without the retromer-stabilizing agent.

4. The complex of claim 3, wherein the thermal stability of the complex is increased by at least about 6° C., about 7° C., about 8° C., about 9° C., or about 10° C., as compared to that of retromer without the retromer-stabilizing agent.

5. The complex of claim 2, wherein the retromer comprises at least one retromer component that contains at least one mutation.

6. The complex of claim 5, wherein said at least one retromer component is VPS35, VPS29, VPS26 or combination thereof.

7. A method for stabilizing retromer in a cell, the method comprising a step of:
contacting a cell expressing retromer components with a retromer-stabilizing agent in an amount effective to increase the stability of retromer complex,
wherein the retromer-stabilizing agent is a compound of Formula I: 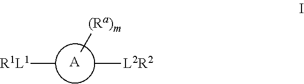
or a pharmaceutically acceptable salt thereof, wherein:
Ring A is an optionally substituted bivalent 3-8 membered saturated, partially unsaturated, or aryl monocyclic ring having 0-4 heteroatoms independently selected from nitrogen, oxygen, or sulfur, or an optionally substituted bivalent 8-10 membered saturated, partially unsaturated, or aryl bicyclic ring having 0-4 heteroatoms independently selected from nitrogen, oxygen, or sulfur;
m is 0-5;
each R^{a} is independently —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂;
each R is independently deuterium, hydrogen, halogen, an optionally substituted C₁₋₆ aliphatic group, or an optionally substituted 3-8 membered saturated, partially unsaturated, or aryl ring having 0-4 heteroatoms independently selected from nitrogen, oxygen, or sulfur, or wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur, or wherein:
two R on the same carbon are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur; and
L¹ and L² are each independently a valence bond or a bivalent optionally substituted C₁₋₁₀ alkylene chain wherein one, two, or three methylene units are optionally and independently replaced by —O—, —N(R)—, —S—, —C(O)—, —C(═NR)— —OC(O)—, —C(O)O—, —OC(O)O—, —S(O)—, —S(O)₂—, —OSO₂O—, —N(R)C(O)—, —C(O)N(R)—, —N(R)C(O)O—, —OC(O)NR—, —N(R)C(O)NR—, and wherein L¹ and L² are each independently optionally substituted with 1-6 R groups; and
R¹ and R² are each independently selected from —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂, —SC(═NR)N(R)₂, or an optionally substituted C₁₋₂₀ aliphatic group.

8. The method of claim 7, wherein the cell expresses a VPS35 mutant polypeptide containing one or more mutations that destabilize retromer.

9. The method of 8, wherein said one or more mutations occur at amino acid residue(s) 534, 541, 579, 582, 586, 589, 629, 630, 633, 637, 672, 675, 725, 729, 769, 772, 776 or any combinations thereof.

10. A method for treating amyloidosis, the method comprising a step of:
administering to a subject having or susceptible to developing amyloidosis a pharmaceutical composition comprising a retromer-stabilizing agent and a pharmaceutically acceptable carrier, wherein the retromer-stabilizing agent is a compound of Formula I: 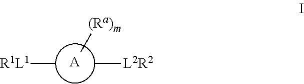
or a pharmaceutically acceptable salt thereof, wherein:
Ring A is an optionally substituted bivalent 3-8 membered saturated, partially unsaturated, or aryl monocyclic ring having 0-4 heteroatoms independently selected from nitrogen, oxygen, or sulfur, or an optionally substituted bivalent 8-10 membered saturated, partially unsaturated, or aryl bicyclic ring having 0-4 heteroatoms independently selected from nitrogen, oxygen, or sulfur;
m is 0-5;
each R^{a} is independently —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂;
each R is independently deuterium, hydrogen, halogen, an optionally substituted C₁₋₆ aliphatic group, or an optionally substituted 3-8 membered saturated, partially unsaturated, or aryl ring having 0-4 heteroatoms independently selected from nitrogen, oxygen, or sulfur, or wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur, or wherein:
two R on the same carbon are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur; and
L¹ and L² are each independently a valence bond or a bivalent optionally substituted C₁₋₁₀ alkylene chain wherein one, two, or three methylene units are optionally and independently replaced by —O—, —N(R)—, —S—, —C(O)—, —C(═NR)— —OC(O)—, —C(O)O—, —OC(O)O—, —S(O)—, —S(O)₂—, —OSO₂O—, —N(R)C(O)—, —C(O)N(R)—, —N(R)C(O)O—, —OC(O)NR—, —N(R)C(O)NR—, and wherein L¹ and L² are each independently optionally substituted with 1-6 R groups; and
R¹ and R² are each independently selected from —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂, —SC(═NR)N(R)₂, or an optionally substituted C₁₋₂₀ aliphatic group.

11. A method for reducing amyloid accumulation in a subject, the method comprising a step of:
administering to a subject having amyloid accumulation a pharmaceutical composition comprising a retromer-stabilizing agent and a pharmaceutically acceptable carrier, in an amount effective to reduce amyloid accumulation in the subject, wherein the retromer-stabilizing agent is a compound of Formula I: 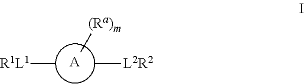
or a pharmaceutically acceptable salt thereof, wherein:
Ring A is an optionally substituted bivalent 3-8 membered saturated, partially unsaturated, or aryl monocyclic ring having 0-4 heteroatoms independently selected from nitrogen, oxygen, or sulfur, or an optionally substituted bivalent 8-10 membered saturated, partially unsaturated, or aryl bicyclic ring having 0-4 heteroatoms independently selected from nitrogen, oxygen, or sulfur;
m is 0-5;
each R^{a} is independently —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂;
each R is independently deuterium, hydrogen, halogen, an optionally substituted C₁₋₆ aliphatic group, or an optionally substituted 3-8 membered saturated, partially unsaturated, or aryl ring having 0-4 heteroatoms independently selected from nitrogen, oxygen, or sulfur, or wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur, or wherein:
two R on the same carbon are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur; and
L¹ and L² are each independently a valence bond or a bivalent optionally substituted C₁₋₁₀ alkylene chain wherein one, two, or three methylene units are optionally and independently replaced by —O—, —N(R)—, —S—, —C(O)—, —C(═NR)— —OC(O)—, —C(O)O—, —OC(O)O—, —S(O)—, —S(O)₂—, —OSO₂O—, —N(R)C(O)—, —C(O)N(R)—, —N(R)C(O)O—, —OC(O)NR—, —N(R)C(O)NR—, and wherein L¹ and L² are each independently optionally substituted with 1-6 R groups; and
R¹ and R² are each independently selected from —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂, —SC(═NR)N(R)₂, or an optionally substituted C₁₋₂₀ aliphatic group.

12. The method of claim 11, wherein the subject expresses a VPS35 mutant polypeptide containing one or more mutations.

13. The method of 12, wherein said one or more mutations occur at amino acid residue(s) 534, 541, 579, 582, 586, 589, 629, 630, 633, 637, 672, 675, 725, 729, 769, 772, 776 or any combinations thereof.

14. A method for promoting retromer-mediated endosome-TGN trafficking of a protein in a cell, the method comprising:
contacting a cell expressing retromer components with a retromer-stabilizing agent in an amount effective to promote retromer-mediated trafficking of a protein from the endosome to the TGN of the cell, wherein the retromer-stabilizing agent is a compound of Formula I: 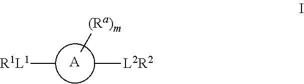
or a pharmaceutically acceptable salt thereof, wherein:
Ring A is an optionally substituted bivalent 3-8 membered saturated, partially unsaturated, or aryl monocyclic ring having 0-4 heteroatoms independently selected from nitrogen, oxygen, or sulfur, or an optionally substituted bivalent 8-10 membered saturated, partially unsaturated, or aryl bicyclic ring having 0-4 heteroatoms independently selected from nitrogen, oxygen, or sulfur;
m is 0-5;
each R^{a} is independently —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂;
each R is independently deuterium, hydrogen, halogen, an optionally substituted C₁₋₆ aliphatic group, or an optionally substituted 3-8 membered saturated, partially unsaturated, or aryl ring having 0-4 heteroatoms independently selected from nitrogen, oxygen, or sulfur, or wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur, or wherein:
two R on the same carbon are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur; and
L¹ and L² are each independently a valence bond or a bivalent optionally substituted C₁₋₁₀ alkylene chain wherein one, two, or three methylene units are optionally and independently replaced by —O—, —N(R)—, —S—, —C(O)—, —C(═NR)— —OC(O)—, —C(O)O—, —OC(O)O—, —S(O)—, —S(O)₂—, —OSO₂O—, —N(R)C(O)—, —C(O)N(R)—, —N(R)C(O)O—, —OC(O)NR—, —N(R)C(O)NR—, and wherein L¹ and L² are each independently optionally substituted with 1-6 R groups; and
R¹ and R² are each independently selected from —R, —CN, —OR, a suitably protected hydroxyl group, —SR, a suitably protected thiol group, —S(O)R, —SO₂R, —OSO₂R, —N(R)₂, a suitably protected amino group, —N(R)C(O)R, —N(R)C(O)C(O)R, —N(R)C(O)N(R)₂, —N(R)C(O)OR, —C(O)OR, —OC(O)R, —C(O)N(R)₂, —OC(O)N(R)₂, —SC(═NR)N(R)₂, or an optionally substituted C₁₋₂₀ aliphatic group.

15. The method of claim 14, wherein the retromer-stabilizing agent binds to a binding site of a VPS35/VPS29 binary complex,
wherein the binding site is at an interface of the VPS35/VPS29 binary complex in a crystal structure; and,
wherein the interface involves one or more of the following amino acid residues corresponding to wild type VPS29: D8, H10, R14, N39, D62, F63, H86, I91, P92, W93, G94, L101, R104, H115, Y139, A141 and L142.

16. The method of claim 14, wherein the retromer-stabilizing agent binds to a binding site of a VPS35/VPS29 binary complex,
wherein the binding site is at an interface of the VPS35/VPS29 binary complex in a crystal structure; and,
wherein the interface involves one or more of the following amino acid residues corresponding to wild type VPS35: F534, F541, L579, R582, Q586, L589, T629, L630, G633, R637, T672, H675, N725, Y729, H769, N772 and H776.

17. The method of claim 14, wherein the retromer-stabilizing agent is:

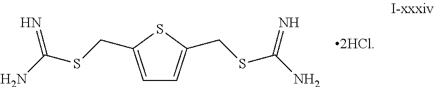 [SMILES: N=C(N)SCc1ccc(CSC(=N)N)s1]

18. The method of claim 14, wherein the cell is a neuronal cell.

19. The method of claim 18, wherein the neuronal cell is a hippocampal neuron.

20. The method of claim 19, wherein the hippocampal neuron is localized to the entorhinal cortex.

21. The method of claim 14, wherein the protein is a type 1 transmembrane receptor.

22. The method of claim 21, wherein the type 1 transmembrane receptor contains a VPS10-domain.

23. The method of claim 21, wherein the type 1 transmembrane receptor is sorLA, sortilin, SorCS1, SorCS2, SorCS3, APP, BASE1, presenilin, Wnt signaling protein or any combination thereof.
